(19) United States
(12) Patent Application Publication
(10) Pub. No.: US 2025/0019445 A1
(43) Pub. Date: Jan. 16, 2025

(54) P-SELECTIN INHIBITION FOR TREATMENT OF CANCER

(71) Applicant: Ramot at Tel-Aviv University Ltd., Tel-Aviv (IL)

(72) Inventors: Ronit SATCHI-FAINARO, Tel-Aviv (IL); Eilam YEINI, Tel-Aviv (IL)

(73) Assignee: Ramot at Tel-Aviv University Ltd., Tel-Aviv (IL)

(21) Appl. No.: 18/885,825

(22) Filed: Sep. 16, 2024

Related U.S. Application Data
(63) Continuation of application No. PCT/IL2023/050279, filed on Mar. 16, 2023.
(60) Provisional application No. 63/320,310, filed on Mar. 16, 2022.

(51) Int. Cl. C07K 16/28; A61K 39/00; A61P 35/00

(52) U.S. Cl. CPC C07K 16/2854; A61P 35/00; C07K 16/2818; A61K 2039/545

(57) ABSTRACT

A method of treating brain-metastasized cancer in a subject in need thereof is disclosed. The method comprises administering to the subject a therapeutically effective amount of an agent that specifically decreases an amount and/or activity of P-selectin and an immunomodulatory agent. Methods of treating additional cancers are also disclosed.

### RELATED APPLICATIONS

This application is a Continuation of PCT Patent Application No. PCT/IL2023/050279 having International filing date of Mar. 16, 2023, which claims the benefit of priority under 35 USC § 119 (e) of U.S. Provisional Patent Application No. 63/320,310 filed on Mar. 16, 2022. The contents of the above applications are all incorporated by reference as if fully set forth herein in their entirety.

### FIELD AND BACKGROUND OF THE INVENTION

The present invention, in some embodiments thereof, relates to a method of treating brain metastasized cancers and glioblastoma by selectively decreasing the activity or amount of P-selectin.

Primary and secondary brain malignancies affect many patients worldwide which, in most cases, have no effective treatments. Among primary brain tumors, glioblastoma (GB) is the most lethal and common type, exhibiting highly heterogenic, invasive and aggressive nature. Brain metastases affect 10-20% of all cancer patients leading to high mortality rates. CNS tumors possess great therapeutic challenges, partially due to the unique environment of the brain which exhibits highly suppressive environment, low immune infiltration and the lack of success of current targeted- and immuno-therapies. Among the brain microenvironment cells, the microglia were shown to facilitate CNS tumors' invasion and immunosuppression. However, the reciprocal mechanisms by which tumor cells alter microglia/macrophages behavior are not fully understood.

It has been previously shown that P-selectin (SELP) mediates microglia-enhanced GB proliferation and invasion by altering microglia/macrophages activation state (Yeini et al., Nature Communications 2021, 12 (1), 1912).

Additional background art includes PCT Application No. WO2022/059008, U.S. patent application No. 20200171064 (which teaches isoquercetin or quercetin for treatment of cancer, including glioblastoma) and U.S. patent application No. 20190241665 (which teaches P-selectin inhibitors for the treatment of metastasized cancers).

Shamay et al., Science Translational Medicine, Volume 8, issue 345, 29 Jun. 2016 teaches that P-selectin is expressed on cancer cells in several human tumor types.

Ferber et al., eLife 2017;6: e25281. DOI: www (dot) doi (dot) org/10 (dot) 7554/eLife (dot) 252 81 teaches that P-selectin is not only expressed on tumor endothelium but also on glioblastoma cells and may be used as a target for selective delivery of anti-cancer agents.

### SUMMARY OF THE INVENTION

According to an aspect of the present invention there is provided a method of treating brain-metastasized cancer in a subject in need thereof comprising administering to the subject a therapeutically effective amount of an agent that specifically decreases an amount and/or activity of P-selectin and an immunomodulatory agent, thereby treating the brain metastasized cancer.

According to an aspect of the present invention there is provided a method of treating pancreatic or kidney cancer in a subject in need thereof comprising administering to the subject a therapeutically effective amount of an agent that specifically decreases an amount and/or activity of P-selectin, thereby treating the pancreatic or kidney cancer.

According to an aspect of the present invention there is provided a method of treating a cancer selected from the group consisting of pancreatic cancer, lung cancer, breast cancer, primary melanoma and kidney cancer in a subject in need thereof comprising administering to the subject a therapeutically effective amount of an agent that specifically decreases an amount and/or activity of P-selectin and an immunomodulatory agent, thereby treating the cancer.

According to embodiments of the invention, the brain-metastasized cancer is brain-metastasized melanoma, brain-metastasized breast cancer, brain-metastasized lung cancer, or brain-metastasized colorectal cancer.

According to embodiments of the invention, the agent that specifically decreases an amount and/or activity of P-selectin specifically binds to P-selectin or a polynucleotide encoding the P-selectin.

According to embodiments of the invention, the agent that specifically decreases an amount and/or activity of P-selectin binds to P-Selectin glycoprotein ligand-1 (PSGL-1) or a polynucleotide encoding the PSGL-1.

According to embodiments of the invention, the method further comprises administering to the subject an immunomodulatory agent.

According to embodiments of the invention, the immunomodulatory agent comprises an immunomodulatory antibody.

According to embodiments of the invention, the immunomodulatory antibody is selected from the group consisting of anti-CTLA4, anti-CD40, anti-41BB, anti-OX40, anti-PD1, anti-PDL1, anti-LAG3, anti-IDO, and anti-TIGIT.

According to embodiments of the invention, the agent that specifically decreases an amount and/or activity of P-selectin is an inhibitory antibody that binds to and inhibits the P-selectin.

According to embodiments of the invention, the inhibitory antibody is Crizanlizumab or Inclacumab.

According to embodiments of the invention, a dose of the Crizanlizumab is about 5 mg/kg once every two weeks or once every four weeks.

According to embodiments of the invention, the inhibitory antibody is attached to a therapeutic agent.

According to embodiments of the invention, the inhibitory antibody is not attached to a therapeutic agent.

According to embodiments of the invention, the agent that specifically decreases an amount and/or activity of P-selectin is a small molecule agent.

According to embodiments of the invention, the agent that specifically decreases an amount and/or activity of P-selectin is a polynucleotide agent.

According to embodiments of the invention, the agent that specifically decreases an amount and/or activity of P-selectin is co-formulated with the immunomodulatory agent.

According to embodiments of the invention, the agent that specifically decreases an amount and/or activity of P-selectin is comprised in a nanoparticle.

According to embodiments of the invention, the nanoparticle is attached to a targeting moiety that increases delivery across the blood brain barrier.

According to embodiments of the invention, the agent that specifically decreases an amount and/or activity of P-selectin is attached to a targeting moiety that increases delivery across the blood brain barrier.

According to an aspect of the present invention, there is provided a method of treating glioblastoma or brain-metastasized melanoma in a subject in need thereof comprising administering to the subject a therapeutically effective amount of Crizanlizumab and an anti-PD1 antibody, thereby treating the glioblastoma or brain metastasized melanoma.

According to embodiments of the invention, the Crizanlizumab is delivered once every two weeks or once every four weeks at a dose of about 5 mg/kg.

According to embodiments of the invention, the anti-PD1 antibody is selected from the group consisting of Pembrolizumab (Keytruda), Nivolumab (Opdivo), Cemiplimab (Libtayo) and Dostarlimab (Jemperli).

According to embodiments of the invention, the anti-PD1 antibody is Nivolumab.

According to embodiments of the invention, the Crizanlizumab and the anti-PD1 antibody are initially administered once every two weeks for a duration of at least four weeks.

According to embodiments of the invention, a dose of the anti-PD1 antibody, is about 3 mg/kg once every two weeks.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fec.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced. In the drawings:

FIGS. 1A-C. PD-1/PD-L1 are highly expressed in glioblastoma patient tissues, and their expression is correlated with SELP/PSGL-1 expression. Analysis of glioblastoma databases showing high expression of PD-1 (PDCD1) and PD-L1 (CD207) in GB samples compared to healthy human brain tissues and low-grade glioma (A-B), and positive correlation between PSGL-1 (SELPLG) expression to PD-1 and PD-L1 expression, and between SELP expression to PD-1 expression. The analysis was performed using the GlioVis data portal.

FIGS. 2A-B. SELP, PSGL-1, PD-1, and PD-L1 are co-expressed in multiple patient BM and DIPG samples. A. Immunostaining showing high expression of SELP in melanoma, breast, lung, and CRC BM as well as in DIPG patient samples. PSGL-I was found to be highly expressed in melanoma, breast and lung BM, and in DIPG patient samples. B. Immunostaining showing high expression of PD-1 in melanoma and lung BM and high expression of PD-L1 in melanoma, breast, lung, and CRC BM and in DIPG patient samples. All samples were compared to normal human brain tissue which showed low expression of all the stained markers.

FIGS. 3A-B. SELP, PSGL-1, PD-1, and PD-L1 are co-expressed in various patient primary tumor samples. A. Immunostaining shows high expression of SELP and PSGL-1 in primary melanoma, breast, and lung patient samples and high expression of SELP in primary PDAC patient samples. B. Immunostaining shows high expression of PD-1 and PD-L1 in primary melanoma, PDAC and lung patient samples. 10 FIGS. 4A-C. Microglia are activated in melanoma brain metastasis in vivo and facilitate melanoma cell proliferation and invasion in vitro. A. Iba-1 Immunostaining showing positive staining for activated microglia in the tumor site using patient-derived MBM mouse model and patient FFPE samples. B. Co-culture proliferation assay showing increased proliferation of murine RET melanoma cells following the addition of primary murine microglia cells in a concentration-dependent manner. C. 3D spheroid invasion assay showed increased growth and invasion of murine D4M melanoma cells when BV2 murine microglia were incorporated into the spheroids.

FIGS. 5A-C. SELP is highly expressed by melanoma cells in 2D cultures and 3D spheroids. A. Flow cytometry analysis shows high expression of SELP in 2D cultured human A375 and murine B16-F10 and Ret melanoma cell lines. B-C. Flow cytometry analysis showing overexpression of SELP in WM115 (B) and D4M.3A (C) 3D tumor spheroids compared to 2D cultures.

FIGS. 6A-B. Combined treatment with SELP inhibitor and anti-PD-1 generates anti-cancer activity of splenocytes against D4M.3A melanoma spheroids in the presence of microglia. A-B. D4M.3A spheroids composed of cancer cells only (A) or cancer cells and primary murine microglia (B) were co-cultured with freshly-isolated mouse splenocytes. Spheroids were untreated or treated with anti-PD-1 antibody, SELP inhibitor (SELPi), or a combination. The combined treatments of SELPi with anti-PD-1 showed the highest spheroid growth inhibition compared to SELPi or anti-PD-1 mono-treatments.

FIGS. 7A-B. The SELP axis mediates breast cancer spheroid invasion. A. Flow cytometry analysis shows high expression of SELP in murine BRCA-mutated EMT6 breast cancer 3D spheroids. B. Human MDA-BM-231 3D spheroids showed increased invasion capabilities when co-cultured with brain microenvironment cells, and reduced invasion following treatment with SELP inhibitor (SELPi).

FIGS. 8A-D. SELP has a role in lung cancer-microglia interactions and mediates lung cancer spheroid growth. A. Human A549 spheroid growth was increased in presence of human microglia compared to spheroids composed of A549 cells only. B. A549 cells expressed high levels of SELP, while human microglia expressed high levels of PSGL-1 when co-cultured in 3D spheroids, as shown by flow cytometry analysis. C. Treatment with SELP inhibitor (SELPi) inhibits A549 spheroid growth, in the presence of human microglia. D. Treatment with SELPi reduced A549 spheroid growth and invasion in Matrigel, in a dose-dependent manner.

### DESCRIPTION OF SPECIFIC EMBODIMENTS OF THE INVENTION

The present invention, in some embodiments thereof, relates to a method of treating brain metastasized cancers and glioblastoma by selectively decreasing the activity or amount of P-selectin.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

It has recently been found that SELP is overexpressed by melanoma brain metastasis (MBM) cells and tissues, both in patient-derived samples and murine mouse models. 40% to 50% in patients with melanoma stage IV will experience brain metastasis. The combination of anti-CTLA-4 antibody (Ipilimumab) and anti-PD-1 antibody (Keytruda (pembrolizumab), Opdivo (nivolumab), Libtayo (cemiplimab)) has been proven effective in patients with melanoma brain metastasis with up to 50% response rate in non-symptomatic patients. However, heterogeneity in immune responses and resistance to treatments are frequently observed due to distinct adaptive and innate immune cells infiltration into the tumor.

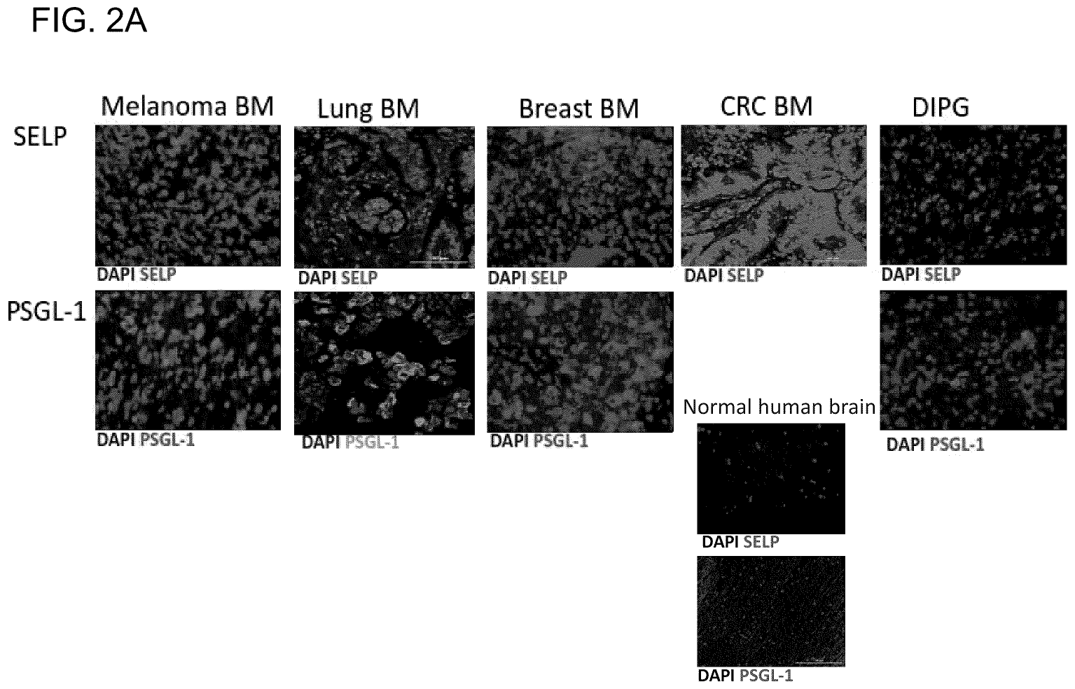

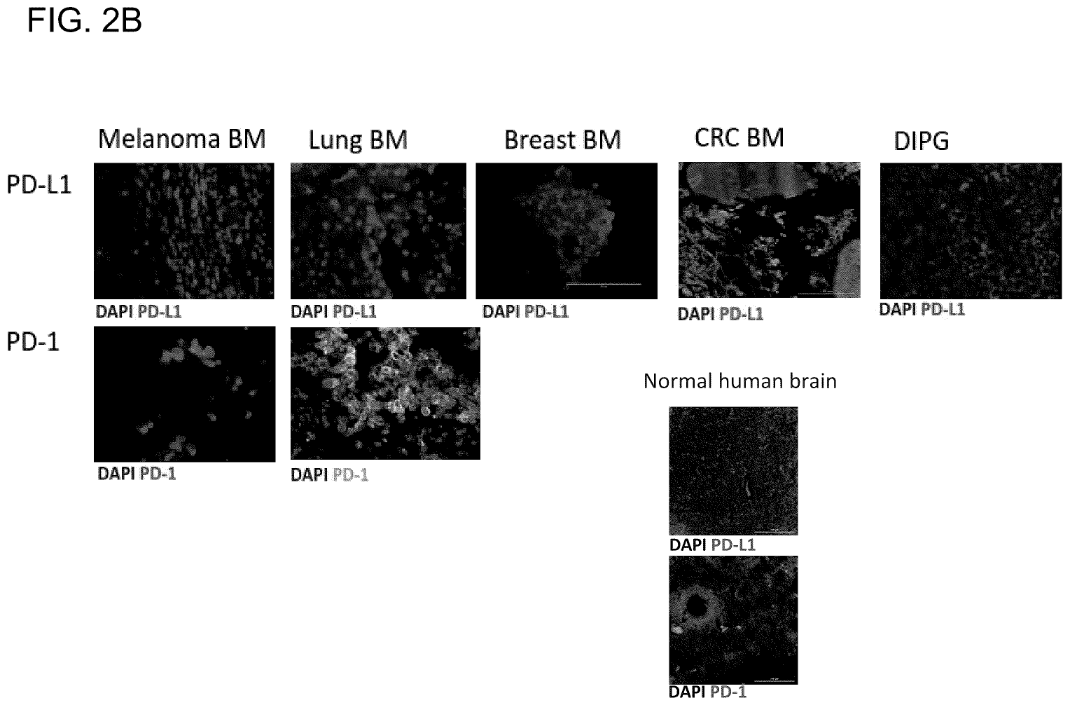

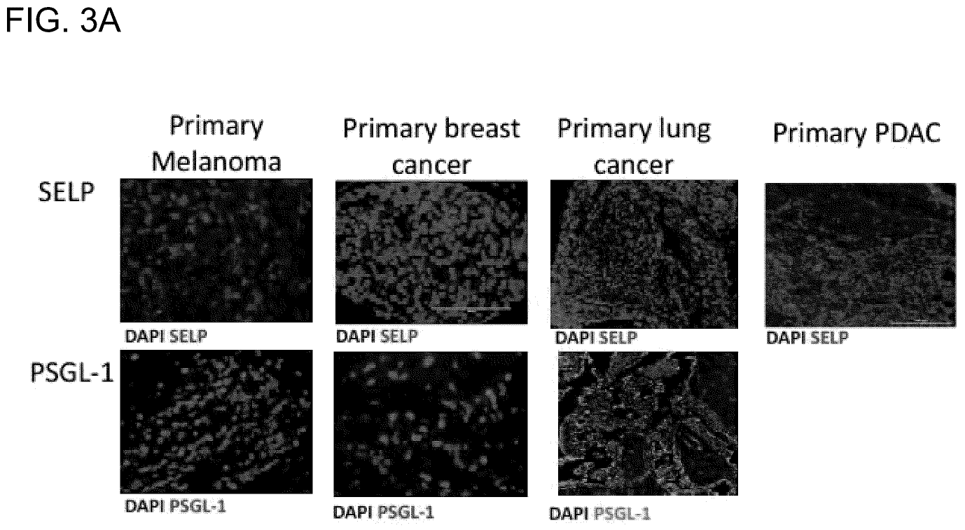

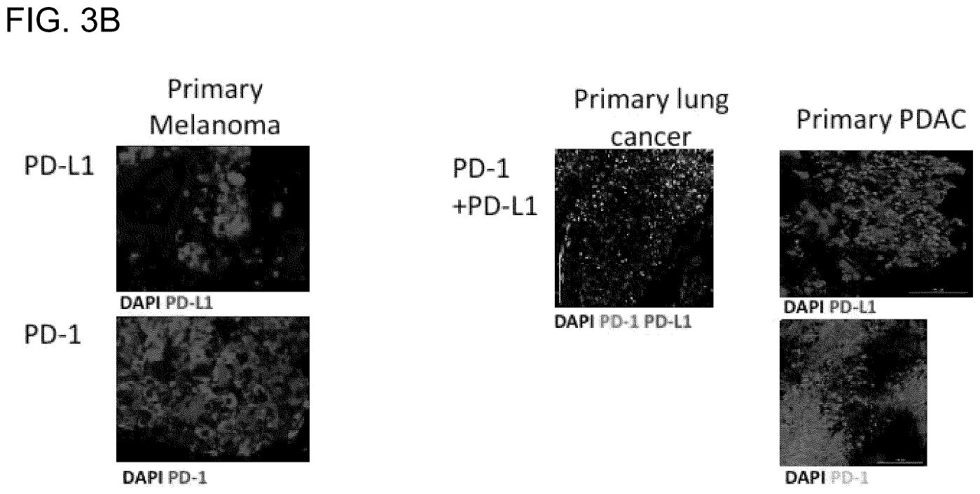

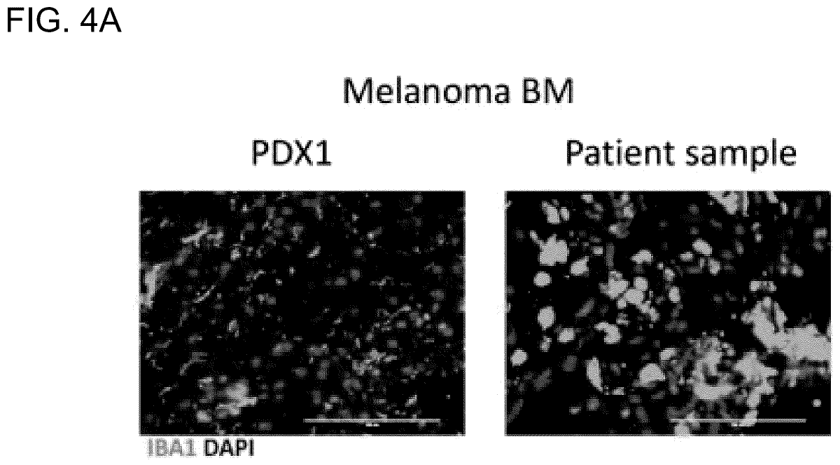

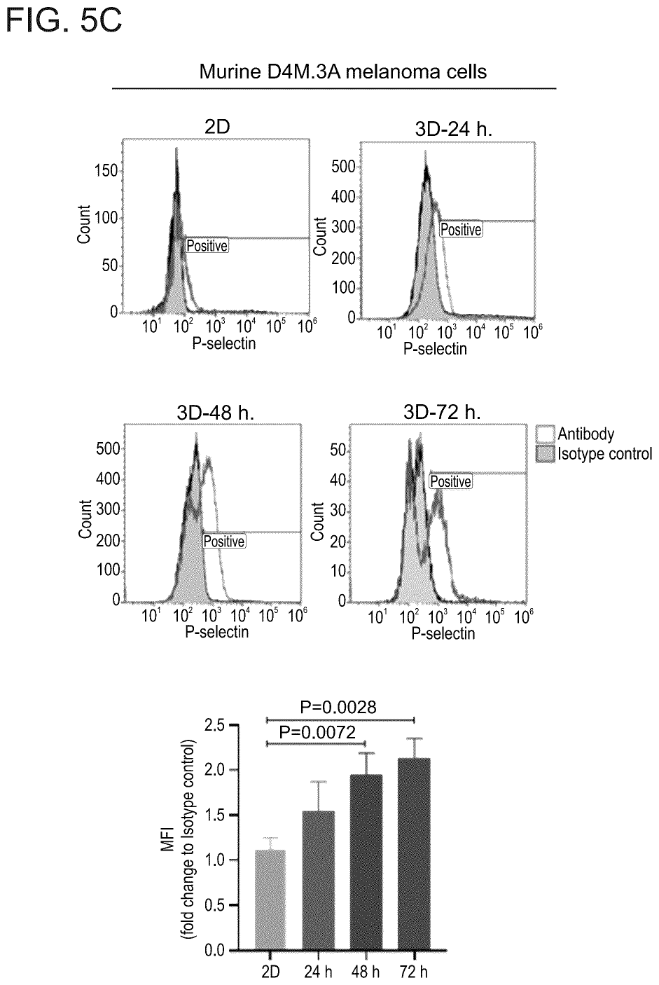

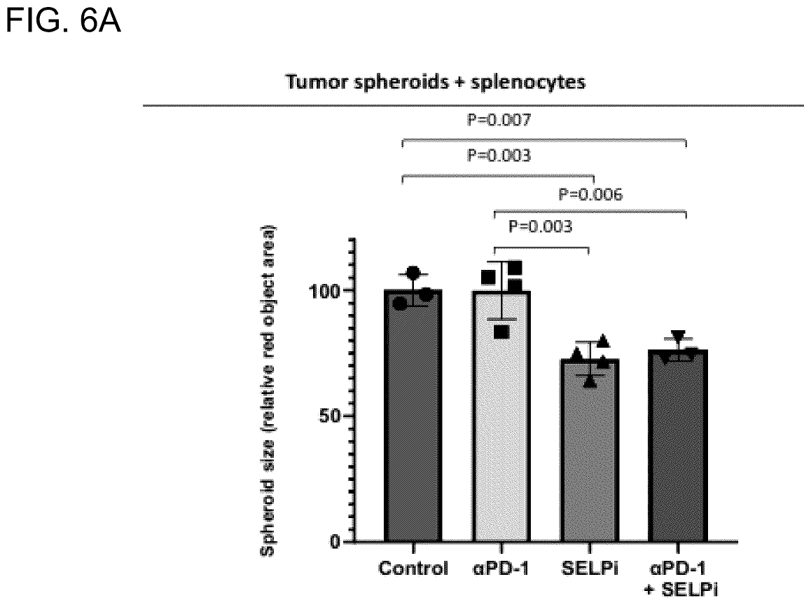

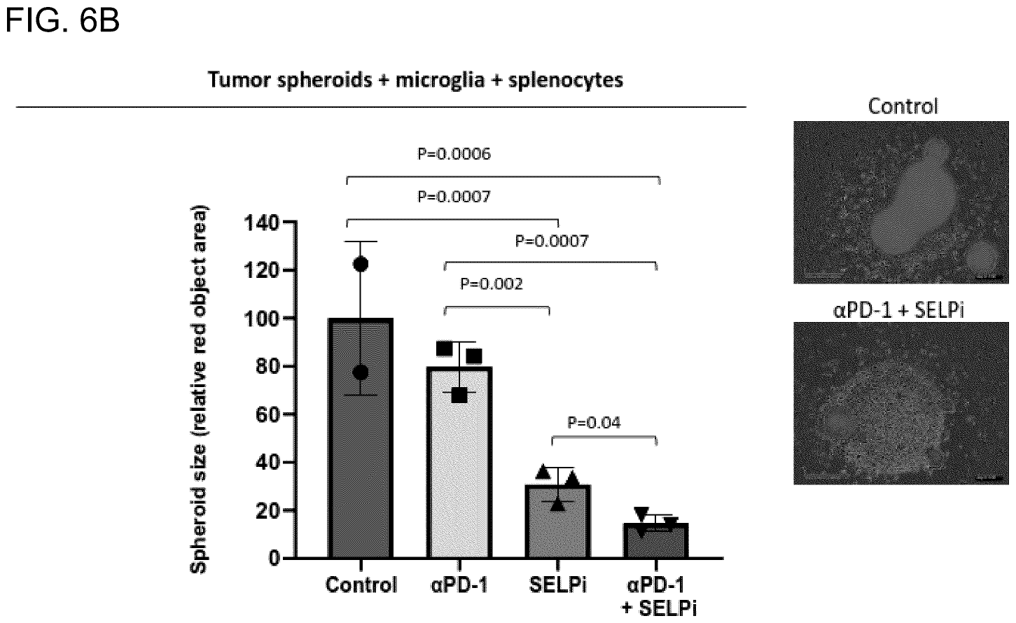

The present inventors have now demonstrated that combining anti-PD1 (or anti-PD-L1, e.g. Tecentriq (atezolizumab), Bavencio (avelumab) and Imfinzi (durvalumab), or small molecule inhibitors of PD-L1) with anti-SELP (antibody or small molecules) sensitizes the tumors to immunotherapy (FIGS. 6A-B). Accordingly, the present inventors propose that SELP inhibition in combination with immunotherapies such as checkpoint modulators, vaccines or CAR T therapies, has therapeutic potential for primary and secondary brain malignancies patients as well as for SELP-expressing primary tumors such as: PDAC/pancreas, RCC/Kidney, Melanoma/skin, and lung (see FIGS. 2A-B and FIGS. 3A-B).

Thus, according to an aspect of the present invention there is provided a method of treating brain-metastasized cancer in a subject in need thereof comprising administering to the subject a therapeutically effective amount of an agent that specifically decreases an amount and/or activity of P-selectin and an immunomodulatory agent, thereby treating the brain metastasized cancer.

According to another aspect of the present invention there is provided a method of treating pancreatic or kidney cancer in a subject in need thereof comprising administering to the subject a therapeutically effective amount of an agent that specifically decreases an amount and/or activity of P-selectin, thereby treating the pancreatic or kidney cancer.

According to still another aspect of the present invention there is provided a method of treating a cancer selected from the group consisting of pancreatic cancer, lung cancer, breast cancer, primary melanoma and kidney cancer in a subject in need thereof comprising administering to the subject a therapeutically effective amount of an agent that specifically decreases an amount and/or activity of P-selectin and an immunomodulatory agent, thereby treating the cancer.

As used herein, the term “subject” refers to a mammal, such as a rodent, a feline, a canine, and a primate. Preferably, a subject according to the invention is a human. In one embodiment, the subject is a non-operable and non-irradiable subject. In one embodiment, the subject has a tumor comprising two or more lobes.

P-selectin is a member of the selectin family of adhesion glycoproteins which also includes L- and E-selectins. The selectins mediate the recruitment, initial tethering and rolling, and adherence of leukocytes to sites of inflammation. P-selectin is stored in Weibel-Palade bodies of endothelial cells and alpha-granules of platelets and is rapidly mobilized to the plasma membrane upon stimulation by vasoactive substances such as histamine and thrombin.

P-selectin is a transmembrane glycoprotein (SwissProt sequence P16109) composed of an NH₂-terminal lectin domain, followed by an epidermal growth factor (EGF)-like domain and nine consensus repeat domains. It is anchored in the membrane by a single transmembrane domain and contains a small cytoplasmic tail.

Human P-selectin (also referred to as SELP) has a Uniprot number P16109 and a REFSEQ mRNA NM_003005.4.

P-selectin plays its central role in the recruitment of leukocytes to inflammatory and thrombotic sites by binding to its counter-receptor, P-selectin glycoprotein ligand-1 (PSGL-1) (or a PSGL-1-like receptor on sickled red blood cells), which is a mucin-like glycoprotein constitutively expressed on leukocytes including neutrophils, monocytes, platelets, and on some endothelial cells.

Human PSGL-1 has a Uniprot Number Q14242 and REFSEQ mRNA as set forth in NM_001206609.2 or NM_003006.4.

Thus, the present invention contemplates down-regulating the function of P-selectin by using (1) antibodies to P-selectin, (2) antibodies to PSGL-1, (3) small molecules that mimic the binding domain of PSGL-1, and (4) other molecules that disrupt the binding of P-selectin to PSGL-1. Such agents are further described herein below.

In another embodiment, the agent down-regulates the amount of P-selectin, by reducing expression of P-selectin.

In still another embodiment, the agent down-regulates expression of PSGL-1.

As used herein the phrase “downregulates expression” refers to downregulating the expression of P-selectin or PSGL-1 at the genomic (e.g. homologous recombination and site specific endonucleases) and/or the transcript level using a variety of molecules which interfere with transcription and/or translation (e.g., RNA silencing agents, CRISPR/Cas-9) or on the protein level (e.g., aptamers, small molecules and inhibitory peptides, antagonists, enzymes that cleave the polypeptide, antibodies and the like).

For the same culture conditions, the expression is generally expressed in comparison to the expression in a cell of the same species but not contacted with the agent or contacted with a vehicle control, also referred to as control.

Down regulation of expression may be either transient or permanent.

According to specific embodiments, down regulating expression refers to the absence of mRNA and/or protein, as detected by RT-PCR or Western blot, respectively.

According to other specific embodiments down regulating expression refers to a decrease in the level of mRNA and/or protein, as detected by RT-PCR or Western blot, respectively. The reduction may be by at least a 10%, at least 20%, at least 30%, at least 40%, at least 50%, at least 60%, at least 70%, at least 80%, at least 90%, at least 95% or at least 99% reduction.

Non-limiting examples of agents capable of down regulating P-selectin or PSGL-1 expression are described in details hereinbelow.

### Down-Regulation at the Nucleic Acid Level

Down-regulation at the nucleic acid level is typically effected using a nucleic acid agent, having a nucleic acid backbone, DNA, RNA, mimetics thereof or a combination of same. The nucleic acid agent may be encoded from a DNA molecule or provided to the cell per se.

According to specific embodiments, the downregulating agent is a polynucleotide.

According to specific embodiments, the downregulating agent is a polynucleotide capable of hybridizing to a gene or mRNA encoding P-selectin.

According to specific embodiments, the downregulating agent is a polynucleotide capable of hybridizing to a gene or mRNA encoding PSGL-1.

According to specific embodiments, the downregulating agent directly interacts with P-selectin.

According to specific embodiments, the agent directly binds to P-selectin.

According to specific embodiments, the agent indirectly binds P-selectin (e.g. binds an effector of P-selectin).

According to specific embodiments the downregulating agent is an RNA silencing agent or a genome editing agent.

Thus, downregulation of P-selectin or PSGL-1 can be achieved by RNA silencing.

As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. The RNA silencing agent may be administered as a naked oligonucleotide, may comprise modified bases, may be a stabilized oligonucleotide, entrapped or conjugated to a nanoparticle or any nanocarrier (polymers, lipids-LNP, liposomes, micelles, etc.)-see for example Scomparin et al Biotechnology Advances, Volume 33, Issue 6, Part 3, Nov. 1, 2015, Pages 1294-1309. In one embodiment, the RNA silencing agent is capable of inducing RNA interference.

In another embodiment, the RNA silencing agent is capable of mediating translational repression.

According to an embodiment of the invention, the RNA silencing agent is specific to the target RNA (e.g., P-selectin) and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

RNA interference refers to the process of sequence-specific post-transcriptional gene silencing in animals mediated by short interfering RNAs (siRNAs).

Following is a detailed description on RNA silencing agents that can be used according to specific embodiments of the present invention.

DsRNA, siRNA and shRNA—The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplate use of dsRNA to downregulate protein expression from mRNA.

According to one embodiment dsRNA longer than 30 bp are used. Various studies demonstrate that long dsRNAs can be used to silence gene expression without inducing the stress response or causing significant off-target effects-see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

According to some embodiments of the invention, dsRNA is provided in cells where the interferon pathway is not activated, see for example Billy et al., PNAS 2001, Vol 98, pages 14428-14433 and Diallo et al., Oligonucleotides, Oct. 1, 2003, 13 (5): 381-392. doi: 10.1089/154545703322617069.

According to an embodiment of the invention, the long dsRNA are specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly (A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small interfering RNA duplexes (generally between 18-30 base pairs) that induce the RNA interference (RNAi) pathway. Typically, siRNAs are chemically synthesized as 21 mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is suggested to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., a siRNA) may be connected to form a hairpin or stem-loop structure (e.g., a shRNA). Thus, as mentioned, the RNA silencing agent of some embodiments of the invention may also be a short hairpin RNA (shRNA).

miRNA and miRNA mimics-According to another embodiment the RNA silencing agent may be a miRNA.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms and have been shown to play a role in development, homeostasis, and disease etiology.

Antisense—Antisense is a single stranded RNA designed to prevent or inhibit expression of a gene by specifically hybridizing to its mRNA. Downregulation of a P-selectin can be effected using an antisense polynucleotide capable of specifically hybridizing with an mRNA transcript encoding P-selectin. Downregulation of PSGL-I can be effected using an antisense polynucleotide capable of specifically hybridizing with an mRNA transcript encoding PSGL-1.

Design of antisense molecules, which can be used to efficiently downregulate a P-selectin or PSGL-I must be effected while considering two aspects important to the antisense approach. The first aspect is delivery of the oligonucleotide into the cytoplasm of the appropriate cells, while the second aspect is design of an oligonucleotide, which specifically binds the designated mRNA within cells in a way which inhibits translation thereof.

Downregulation can be achieved by inactivating the gene (i.e. the P-selectin or PSGL-1 gene) via introducing targeted mutations involving loss-of function alterations (e.g. point mutations, deletions and insertions) in the gene structure.

As used herein, the phrase “loss-of-function alterations” refers to any mutation in the DNA sequence of a gene which results in downregulation of the expression level and/or activity of the expressed product, i.e., the mRNA transcript and/or the translated protein. Non-limiting examples of such loss-of-function alterations include a missense mutation, i.e., a mutation which changes an amino acid residue in the protein with another amino acid residue and thereby abolishes the enzymatic activity of the protein; a nonsense mutation, i.e., a mutation which introduces a stop codon in a protein, e.g., an early stop codon which results in a shorter protein devoid of the enzymatic activity; a frame-shift mutation, i.e., a mutation, usually, deletion or insertion of nucleic acid(s) which changes the reading frame of the protein, and may result in an early termination by introducing a stop codon into a reading frame (e.g., a truncated protein, devoid of the enzymatic activity), or in a longer amino acid sequence (e.g., a readthrough protein) which affects the secondary or tertiary structure of the protein and results in a non-functional protein, devoid of the enzymatic activity of the non-mutated polypeptide; a readthrough mutation due to a frame-shift mutation or a modified stop codon mutation (i.e., when the stop codon is mutated into an amino acid codon), with an abolished enzymatic activity; a promoter mutation, i.e., a mutation in a promoter sequence, usually 5′ to the transcription start site of a gene, which results in down-regulation of a specific gene product; a regulatory mutation, i.e., a mutation in a region upstream or downstream, or within a gene, which affects the expression of the gene product; a deletion mutation, i.e., a mutation which deletes coding nucleic acids in a gene sequence and which may result in a frame-shift mutation or an in-frame mutation (within the coding sequence, deletion of one or more amino acid codons); an insertion mutation, i.e., a mutation which inserts coding or non-coding nucleic acids into a gene sequence, and which may result in a frame-shift mutation or an in-frame insertion of one or more amino acid codons; an inversion, i.e., a mutation which results in an inverted coding or non-coding sequence; a splice mutation i.e., a mutation which results in abnormal splicing or poor splicing; and a duplication mutation, i.e., a mutation which results in a duplicated coding or non-coding sequence, which can be in-frame or can cause a frame-shift.

According to specific embodiments loss-of-function alteration of a gene may comprise at least one allele of the gene.

The term “allele” as used herein, refers to any of one or more alternative forms of a gene locus, all of which alleles relate to a trait or characteristic. In a diploid cell or organism, the two alleles of a given gene occupy corresponding loci on a pair of homologous chromosomes.

According to other specific embodiments, loss-of-function alteration of a gene comprises both alleles of the gene. In such instances, the P-selectin or PSGL-1 may be in a homozygous form or in a heterozygous form.

Methods of introducing nucleic acid alterations to a gene of interest are well known in the art [see for example Menke D. Genesis (2013) 51:-618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entiretics| and include targeted homologous recombination, site specific recombinases, PB transposases and genome editing by engineered nucleases. Agents for introducing nucleic acid alterations to a gene of interest can be designed publically available sources or obtained commercially from Transposagen, Addgene and Sangamo Biosciences.

Following is a description of various exemplary methods used to introduce nucleic acid alterations to a gene of interest and agents for implementing same that can be used according to specific embodiments of the present invention.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDR) and non-homologous end-joining (NHEJ). NHEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, MT et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8, 148,098; or 8, 163,514, the contents of each are incorporated herein by reference in their entirety. Alternatively, meganucleases with site-specific cutting characteristics can be obtained using commercially available technologies e.g., Precision Biosciences' Directed Nuclease Editor™ genome editing technology.

ZFNs and TALENs Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the FokI domains heterodimerize to create a double-stranded break. Repair of these double-stranded breaks through the non-homologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lec et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, CA).

Method for designing and obtaining TALENs are described in e.g. Reyon et al. Nature Biotechnology 2012 May;30 (5): 460-5; Miller et al. Nat Biotechnol. (2011) 29:143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www (dot) talendesign (dot) org). TALEN can also be designed and obtained commercially from e.g., Sangamo Biosciences™ (Richmond, CA).

CRISPR-Cas system-Many bacteria and archaca contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337:816 821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded brakes produced by CRISPR/Cas can undergo homologous recombination or NHEJ.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

Non-limiting examples of gRNA sequences that can be used with some embodiments of the present invention are described in the literature (Sanjana N. E., Shalem O., Zhang F. Nat Methods. 2014 August;11 (8): 783-4) and in the genscript website see www (dot) genscript (dot) com/gRNA-detail/6403/SELP-CRISPR-guide-RNA.

According to specific embodiments, the gRNA sequence does not have a significant off target effect. Methods of determining off target effect are well known in the art, such as BGI Human Whole Genome Sequencing (described in Nature;491:65-56.2012), next generation sequencing (NGS) using e.g. commercially available kits such as Alt-R-Genom Editing (IDT detection kit) or Sure select target enrich <1% variant allele frequency (Agilent).

In order to use the CRISPR system, both gRNA and Cas9 should be expressed in a target cell. The insertion vector can contain both cassettes on a single plasmid or the cassettes are expressed from two separate plasmids. CRISPR plasmids are commercially available such as the px330 plasmid from Addgene. Alternatively, the target cell can be transfected with both gRNA and Cas9 without plasmid using e.g. a transfection reagent such as CRISPRMAX [see e.g. Yu et al. (2016) JDIBiotechnol Lett. 38 (6): 919-29]. In some cells electroporation can improve the transfection of the gRNA and the Cas9 [sce e.g. Liang et al. (2015) Journal of Biotechnology 208, 2015, Pages 44-53; and Liang et al. (2017) Journal of Biotechnology, Volume 241, 2017, pp. 136-146].

“Hit and run” or “in-out”-involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, electroporated into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy-involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After electroporation and positive selection, homologously targeted clones are identified. Next, a second targeting vector that contains a region of homology with the desired mutation is electroporated into targeted clones, and negative selection is applied to remove the selection cassette and introduce the mutation. The final allele contains the desired mutation while eliminating unwanted exogenous sequences.

Site-Specific Recombinases—The Cre recombinase derived from the PI bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

Transposases—As used herein, the term “transposase” refers to an enzyme that binds to the ends of a transposon and catalyzes the movement of the transposon to another part of the genome.

As used herein the term “transposon” refers to a mobile genetic element comprising a nucleotide sequence which can move around to different positions within the genome of a single cell. In the process the transposon can cause mutations and/or change the amount of a DNA in the genome of the cell.

A number of transposon systems that are able to also transpose in cells e.g. vertebrates have been isolated or designed, such as Sleeping Beauty [Izsvak and Ivics Molecular Therapy (2004) 9, 147-156], piggyBac [Wilson et al. Molecular Therapy (2007) 15, 139-145], Tol2 [Kawakami et al. PNAS (2000) 97 (21): 11403-11408] or Frog Prince [Miskey et al. Nucleic Acids Res. December 1, (2003) 31 (23): 6873-6881]. Generally, DNA transposons translocate from one DNA site to another in a simple, cut-and-paste manner. Each of these elements has their own advantages, for example, Sleeping Beauty is particularly useful in region-specific mutagenesis, whereas Tol2 has the highest tendency to integrate into expressed genes. Hyperactive systems are available for Sleeping Beauty and piggyBac. Most importantly, these transposons have distinct target site preferences, and can therefore introduce sequence alterations in overlapping, but distinct sets of genes. Therefore, to achieve the best possible coverage of genes, the use of more than one element is particularly preferred. The basic mechanism is shared between the different transposases, therefore we will describe piggyBac (PB) as an example.

PB is a 2.5 kb insect transposon originally isolated from the cabbage looper moth, Trichoplusia ni. The PB transposon consists of asymmetric terminal repeat sequences that flank a transposase, PBase. PBase recognizes the terminal repeats and induces transposition via a “cut-and-paste” based mechanism, and preferentially transposes into the host genome at the tetranucleotide sequence TTAA. Upon insertion, the TTAA target site is duplicated such that the PB transposon is flanked by this tetranucleotide sequence. When mobilized, PB typically excises itself precisely to reestablish a single TTAA site, thereby restoring the host sequence to its pretransposon state. After excision, PB can transpose into a new location or be permanently lost from the genome.

Typically, the transposase system offers an alternative means for the removal of selection cassettes after homologous recombination quit similar to the use Cre/Lox or Flp/FRT. Thus, for example, the PB transposase system involves introduction of a targeting vector with 3′ and 5′ homology arms containing the mutation of interest, two PB terminal repeat sequences at the site of an endogenous TTAA sequence and a selection cassette placed between PB terminal repeat sequences. Positive selection is applied and homologous recombinants that contain targeted mutation are identified. Transient expression of PBase removes in conjunction with negative selection results in the excision of the selection cassette and selects for cells where the cassette has been lost. The final targeted allele contains the introduced mutation with no exogenous sequences.

For PB to be useful for the introduction of sequence alterations, there must be a native TTAA site in relatively close proximity to the location where a particular mutation is to be inserted.

Genome editing using recombinant adeno-associated virus (rAAV) platform—this genome-editing platform is based on rAAV vectors which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross and/or subtle endogenous gene alterations in a cell. rAAV genome editing has the advantage in that it targets a single allele and does not result in any off-target genomic alterations. rAAV genome editing technology is commercially available, for example, the rAAV GENESIS™ system from Horizon™ (Cambridge, UK).

It will be appreciated that the agent can be a mutagen that causes random mutations and the cells exhibiting downregulation of the expression level and/or activity of the target may be selected.

The mutagens may be, but are not limited to, genetic, chemical or radiation agents. For example, the mutagen may be ionizing radiation, such as, but not limited to, ultraviolet light, gamma rays or alpha particles. Other mutagens may include, but not be limited to, base analogs, which can cause copying errors; deaminating agents, such as nitrous acid; intercalating agents, such as cthidium bromide; alkylating agents, such as bromouracil; transposons; natural and synthetic alkaloids; bromine and derivatives thereof; sodium azide; psoralen (for example, combined with ultraviolet radiation). The mutagen may be a chemical mutagen such as, but not limited to, ICR191, 1,2,7,8-diepoxy-octane (DEO), 5-azaC, N-methyl-N-nitrosoguanidine (MNNG) or ethyl methane sulfonate (EMS).

Methods for qualifying efficacy and detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

Sequence alterations in a specific gene can also be determined at the protein level using e.g. chromatography, electrophoretic methods, immunodetection assays such as ELISA and western blot analysis and immunohistochemistry.

In addition, one ordinarily skilled in the art can readily design a knock-in/knock-out construct including positive and/or negative selection markers for efficiently selecting transformed cells that underwent a homologous recombination event with the construct. Positive selection provides a means to enrich the population of clones that have taken up foreign DNA. Non-limiting examples of such positive markers include glutamine synthetase, dihydrofolate reductase (DHFR), markers that confer antibiotic resistance, such as neomycin, hygromycin, puromycin, and blasticidin S resistance cassettes. Negative selection markers are necessary to select against random integrations and/or elimination of a marker sequence (e.g. positive marker). Non-limiting examples of such negative markers include the herpes simplex-thymidine kinase (HSV-TK) which converts ganciclovir (GCV) into a cytotoxic nucleoside analog, hypoxanthine phosphoribosyltransferase (HPRT) and adenine phosphoribosytransferase (ARPT).

### Down-Regulation at the Polypeptide Level

According to specific embodiments the agent capable of downregulating P-selectin is an antibody or antibody fragment capable of specifically binding and inhibiting P-selectin.

Preferably, the antibody specifically binds at least one epitope of P-selectin.

In another embodiment, the agent is an antibody or antibody fragment capable of specifically binding and inhibiting PSGL-1.

Preferably, the antibody specifically binds at least one epitope of PSGL-1.

As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three-dimensional structural characteristics, as well as specific charge characteristics.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

The term “antibody” as used in this invention includes intact molecules as well as functional fragments thereof (that are capable of binding to an epitope of an antigen).

As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three-dimensional structural characteristics, as well as specific charge characteristics.

According to a specific embodiment, the antibody fragments include, but are not limited to, single chain, Fab, Fab′ and F (ab′)₂ fragments, Fd, Fcab, Fv, dsFv, scFvs, diabodies, minibodies, nanobodies, Fab expression library or single domain molecules such as VH and VL that are capable of binding to an epitope of the antigen in an HLA restricted manner.

Suitable antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv Fv (scFv), a disulfide-stabilized Fv (dsFv), an Fab, an Fab′, and an F (ab′) 2, or antibody fragments comprising the Fc region of an antibody.

As used herein, the terms “complementarity-determining region” or “CDR” are used interchangeably to refer to the antigen binding regions found within the variable region of the heavy and light chain polypeptides. Generally, antibodies comprise three CDRs in each of the VH (CDR HI or HI; CDR H2 or H2; and CDR H3 or H3) and three in each of the VL (CDR LI or LI; CDR L2 or L2; and CDR L3 or L3).

The identity of the amino acid residues in a particular antibody that make up a variable region or a CDR can be determined using methods well known in the art and include methods such as sequence variability as defined by Kabat et al. (See, e.g., Kabat et al., 1992, Sequences of Proteins of Immunological Interest, 5th ed., Public Health Service, NIH, Washington D.C.), location of the structural loop regions as defined by Chothia et al. (see, e.g., Chothia et al., Nature 342:877-883, 1989.), a compromise between Kabat and Chothia using Oxford Molecular's AbM antibody modeling software (now Accelrys®, see, Martin et al., 1989, Proc. Natl Acad Sci USA. 86:9268; and world wide web site www (dot) bioinf-org (dot) uk/abs), available complex crystal structures as defined by the contact definition (see MacCallum et al., J. Mol. Biol. 262:732-745, 1996) and the “conformational definition” (see, e.g., Makabe et al., Journal of Biological Chemistry, 283:1156-1166, 2008).

As used herein, the “variable regions” and “CDRs” may refer to variable regions and CDRs defined by any approach known in the art, including combinations of approaches.

Functional antibody fragments comprising whole or essentially whole variable regions of both light and heavy chains are defined as follows:
(i) Fv, defined as a genetically engineered fragment consisting of the variable region of the light chain (VL) and the variable region of the heavy chain (VH) expressed as two chains;
(ii) single chain Fv (“scFv”), a genetically engineered single chain molecule including the variable region of the light chain and the variable region of the heavy chain, linked by a suitable polypeptide linker as a genetically fused single chain molecule.
(iii) disulfide-stabilized Fv (“dsFv”), a genetically engineered antibody including the variable region of the light chain and the variable region of the heavy chain, linked by a genetically engineered disulfide bond.
(iv) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CHI domains thereof;
(v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);
(vi) F (ab′) 2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds);
(vii) Single domain antibodies or nanobodies are composed of a single VH or VL domains which exhibit sufficient affinity to the antigen; and
(viii) Fcab, a fragment of an antibody molecule containing the Fc portion of an antibody developed as an antigen-binding domain by introducing antigen-binding ability into the Fc region of the antibody.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Exemplary methods for generating antibodies employ induction of in-vivo production of antibody molecules, screening of immunoglobulin libraries (Orlandi D. R. et al., 1989. Proc. Natl. Acad. Sci. U.S.A. 86:3833-3837; Winter G. et al., 1991. Nature 349:293-299) or generation of monoclonal antibody molecules by continuous cell lines in culture. These include, but are not limited to, the hybridoma technique, the human B-cell hybridoma technique, and the Epstein-Barr virus (EBV)-hybridoma technique (Kohler G. et al., 1975. Nature 256:495-497; Kozbor D. et al., 1985. J. Immunol. Methods 81:31-42; Cote R J. et al., 1983. Proc. Natl. Acad. Sci. U.S.A 80:2026-2030; Cole SP. et al., 1984. Mol. Cell. Biol. 62:109-120).

In cases where target antigens are too small to elicit an adequate immunogenic response when generating antibodies in-vivo, such antigens (haptens) can be coupled to antigenically neutral carriers such as keyhole limpet hemocyanin (KLH) or serum albumin [e.g., bovine serum albumin (BSA)] carriers (see, for example, U.S. Pat. Nos. 5,189,178 and 5,239,078]. Coupling a hapten to a carrier can be effected using methods well known in the art. For example, direct coupling to amino groups can be effected and optionally followed by reduction of the imino linkage formed. Alternatively, the carrier can be coupled using condensing agents such as dicyclohexyl carbodiimide or other carbodiimide dehydrating agents. Linker compounds can also be used to effect the coupling; both homobifunctional and heterobifunctional linkers are available from Pierce Chemical Company, Rockford, Ill. The resulting immunogenic complex can then be injected into suitable mammalian subjects such as mice, rabbits, and the like. Suitable protocols involve repeated injection of the immunogen in the presence of adjuvants according to a schedule which boosts production of antibodies in the serum. The titers of the immune serum can readily be measured using immunoassay procedures which are well known in the art.

The antisera obtained can be used directly or monoclonal antibodies may be obtained as described hereinabove.

Antibody fragments according to some embodiments of the invention can be prepared by protcolytic hydrolysis of the antibody or by expression in E. coli or mammalian cells (e.g. Chinese hamster ovary cell culture or other protein expression systems) of DNA encoding the fragment.

Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73:119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

As described hereinabove, Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2:97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. Sec, for example, Larrick and Fry [Methods, 2:106-10 (1991)].

As mentioned, the antibody fragment may comprise a Fc region of an antibody termed “Fcab”. Such antibody fragments typically comprise the CH2-CH3 domains of an antibody. Fcabs are engineering to comprise at least one modification in a structural loop region of the antibody, i.e. in a CH3 region of the heavy chain. Such antibody fragments can be generated, for example, as follows: providing a nucleic acid encoding an antibody comprising at least one structural loop region (e.g. Fc region), modifying at least one nucleotide residue of the at least one structural loop regions, transferring the modified nucleic acid in an expression system, expressing the modified antibody, contacting the expressed modified antibody with an epitope, and determining whether the modified antibody binds to the epitope. See, for example, U.S. Pat. Nos. 9,045,528 and 9,133,274 incorporated herein by reference in their entirety.

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhocyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147 (1): 86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368 812-13 (1994); Fishwild et al., Nature Biotechnology 14, 845-51 (1996); Neuberger, Nature Biotechnology 14:826 (1996); and Lonberg and Huszar, Intern. Rev. Immunol. 13, 65-93 (1995).

The antibodies described herein may be conjugated to a therapeutic moiety. The therapeutic moiety can be, for example, a cytotoxic moiety, a toxic moiety, a cytokine moiety and a second antibody moiety comprising a different specificity to the antibodies of the invention.

Non-limiting examples of therapeutic moieties which can be conjugated to the antibody of the invention are provided in Table 1, hereinbelow.

TABLE 1

| | Amino acid sequence | Nucleic acid sequence |
|---|---|---|
| Therapeutic | (GenBank | (GenBank |
| moiety | Accession No.) | Accession No.) |
| Pseudomonas | ABU63124 | EU090068 |
| exotoxin | | |
| Diphtheria toxin | AAV70486 | AY820132.1 |
| interleukin 2 | CAA00227 | A02159 |
| CD3 | P07766 | X03884 |
| CD16 | NP_000560.5 | NM_000569.6 |
| interleukin 4 | NP_000580.1 | NM_000589.2 |
| HLA-A2 | P01892 | K02883 |
| interleukin 10 | P22301 | M57627 |
| Ricin toxin | EEF27734 | EQ975183 |

Other therapeutic moieties that may be attached to the antibody of the invention include anticancer agents such as chemotherapeutic agents including but not limited to tubulin inhibitors, such as exatecan, belotecan, Emtansine, etc.

The therapeutic moiety may be attached or conjugated to the antibody of the invention in various ways, depending on the context, application and purpose.

When the functional moiety is a polypeptide, the immunoconjugate may be produced by recombinant means. For example, the nucleic acid sequence encoding a toxin (e.g., PE38KDEL) or a fluorescent protein [e.g., green fluorescent protein (GFP), red fluorescent protein (RFP) or yellow fluorescent protein (YFP)] may be ligated in-frame with the nucleic acid sequence encoding the antibody of the invention and be expressed in a host cell to produce a recombinant conjugated antibody. Alternatively, the functional moiety may be chemically synthesized by, for example, the stepwise addition of one or more amino acid residues in defined order such as solid phase peptide synthetic techniques.

A functional moiety may also be attached to the antibody of the invention using standard chemical synthesis techniques widely practiced in the art [see e.g., hypertexttransferprotocol://worldwideweb (dot) chemistry (dot) org/portal/Chemistry)], such as using any suitable chemical linkage, direct or indirect, as via a peptide bond (when the functional moiety is a polypeptide), or via covalent bonding to an intervening linker element, such as a linker peptide or other chemical moiety, such as an organic polymer. Chimeric peptides may be linked via bonding at the carboxy (C) or amino (N) termini of the peptides, or via bonding to internal chemical groups such as straight, branched or cyclic side chains, internal carbon or nitrogen atoms, and the like. Description of fluorescent labeling of antibodies is provided in details in U.S. Pat. Nos. 3,940,475, 4,289,747, and 4,376,110.

Exemplary methods for conjugating peptide moieties (therapeutic or detectable moieties) to the antibody of the invention are described herein below:

SPDP conjugation—A non-limiting example of a method of SPDP conjugation is described in Cumber et al. (1985, Methods of Enzymology 112:207-224). Briefly, a peptide, such as a detectable or therapeutic moiety (e.g., 1.7 mg/ml) is mixed with a 10-fold excess of SPDP (50 mM in ethanol); the antibody is mixed with a 25-fold excess of SPDP in 20 mM sodium phosphate, 0.10 M NaCl pH 7.2 and each of the reactions is incubated for about 3 hours at room temperature. The reactions are then dialyzed against PBS. The peptide is reduced, e.g., with 50 mM DTT for 1 hour at room temperature. The reduced peptide is desalted by equilibration on G-25 column (up to 5% sample/column volume) with 50 mM KH₂PO₄ pH 6.5. The reduced peptide is combined with the SPDP-antibody in a molar ratio of 1:10 antibody: peptide and incubated at 4° C. overnight to form a peptide-antibody conjugate.

Glutaraldehyde conjugation—A non-limiting example of a method of glutaraldehyde conjugation is described in G. T. Hermanson (1996, “Antibody Modification and Conjugation, in Bioconjugate Techniques, Academic Press, San Diego). Briefly, the antibody and the peptide (1.1 mg/ml) are mixed at a 10-fold excess with 0.05% glutaraldehyde in 0.1 M phosphate, 0.15 M NaCl pH 6.8, and allowed to react for 2 hours at room temperature. 0.01 M lysine can be added to block excess sites. After—the reaction, the excess glutaraldehyde is removed using a G-25 column equilibrated with PBS (10% v/v sample/column volumes).

Carbodiimide conjugation—Conjugation of a peptide with an antibody can be accomplished using a dehydrating agent such as a carbodiimide, e.g., in the presence of 4-dimethyl aminopyridinc. Carbodiimide conjugation can be used to form a covalent bond between a carboxyl group of peptide and an hydroxyl group of an antibody (resulting in the formation of an ester bond), or an amino group of an antibody (resulting in the formation of an amide bond) or a sulfhydryl group of an antibody (resulting in the formation of a thioester bond). Likewise, carbodiimide coupling can be used to form analogous covalent bonds between a carbon group of an antibody and an hydroxyl, amino or sulfhydryl group of the peptide [see, J. March, Advanced Organic Chemistry: Reaction's, Mechanism, and Structure, pp. 349-50 & 372-74 (3d ed.), 1985]. For example, the peptide can be conjugated to an antibody via a covalent bond using a carbodiimide, such as dicyclohexylcarbodiimide [B. Neises et al. (1978), Angew Chem., Int. Ed. Engl. 17:522; A. Hassner et al. (1978, Tetrahedron Lett. 4475); E. P. Boden et al. (1986, J. Org. Chem. 50:2394) and L. J. Mathias (1979, Synthesis 561)].

According to another embodiment, the antibodies described herein are not conjugated to a therapeutic or a diagnostic moiety.

Another agent which can be used along with some embodiments of the invention to downregulate P-selectin is an aptamer. As used herein, the term “aptamer” refers to double stranded or single stranded RNA molecule that binds to specific molecular target, such as a protein. Various methods are known in the art which can be used to design protein specific aptamers. The skilled artisan can employ SELEX (Systematic Evolution of Ligands by Exponential Enrichment) for efficient selection as described in Stoltenburg R, Reinemann C, and Strehlitz B (Biomolecular engineering (2007) 24 (4): 381-403).

Another agent capable of downregulating P-selectin would be any molecule which binds to and/or cleaves P-selectin. Such molecules can be a small molecule, P-selectin antagonists, or P-selectin inhibitory peptide.

Another contemplated agent which can be used to downregulate P-selectin includes a proteolysis-targeting chimaera (PROTAC). Such agents are heterobifunctional, comprising a ligand which binds to a ubiquitin ligase (such as E3 ubiquitin ligase) and a ligand to P-selectin and optionally a linker connecting the two ligands. Binding of the PROTAC to the target protein leads to the ubiquitination of an exposed lysine on the target protein, followed by ubiquitin proteasome system (UPS)-mediated protein degradation.

In an embodiment, the P-selectin inhibitor is a monoclonal antibody directed towards P-selectin, such as crizanlizumab or inclacumab. These antibodies against P-selectin have been developed to treat sickle cell anemia and myocardial damage following a heart attack, respectively. Both antibodies were well tolerated in patients when administered systemically.

According to a particular embodiment, the P-selectin inhibitor is a monoclonal antibody directed towards PSLGL-1. An example of such an antibody is VTX-0811, which is being developed by Verseau therapeutics (www (dot) verseautx (dot) com/pipeline).

In another embodiment, the P-selectin inhibitor is a small molecule such as rivipansel or tinzaparin, which have been developed to treat sickle cell anemia and as an anticoagulant, respectively. Rivipansel is not specific to P-selectin, but inhibits several members of the selectins family. Tinzaparin is a heparin analogue.

In another embodiment, the P-selectin inhibitor is KF 38789 manufactured by Tocris (3-[7-(2,4-Dimethoxyphenyl)-2,3,6,7-tetrahydro-1,4-thiazepin-5-yl]-4-hydroxy-6-methyl-2H-pyran-2-one).

Additional exemplary P-selectin inhibitors are summarized in Table 2, herein below.

TABLE 2

| Product | | | | |
|---|---|---|---|---|
| name | Product description | Purpose | Phase | References |
| Crizan- | Humanized monoclonal anti-P-selectin antibody. | Treatment of retinal | II | NCT04611880 |
| lizumab | | vasculopathy with | | [1] |
| (SelG1, | | cerebral | | |
| Adakveo) | | leukoencephalopathy | | |
| | | (RVCL) | | |
| | | Study to assess safety | II | NCT01895361 |
| | | and impact of SelG1 | | |
| | | with or without | | |
| | | Hydroxyurea therapy | | |
| | | in Sickle Cell Disease | | |
| | | patients with pain | | |
| | | Crises. | | |
| | | Crizanlizumab for | II | NCT04435184 |
| | | treating COVID-19 | | |
| | | vasculopathy | | |
| | | (CRITICAL). | | |
| PSI-697 | Oral P-selectin inhibitor. | Study evaluating the | I | NCT03860506 |
| | 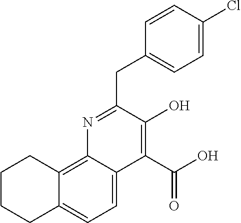 [SMILES: O=C(O)c1c(O)c(Cc2ccc(Cl)cc2)nc2c3c(ccc12)CCCC3] | effects of PSI-697 on platelets in subjects who smoke. | | [2] |
| Cylexin | Synthetic analogue of sialyl Lewis X oligosaccharide. | Cylexin for | II, | NCT00226369 |
| (CY-1503) | 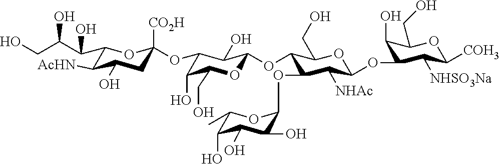 | reduction of reperfusion injury in infant heart surgery. | III | [3] |
| Bimo- | Low-molecular weight sLex-blocking selectin inhibitor that has | Study to evaluate | II | NCT01108913 |
| siamose | been shown to inhibit P-selectin-, E-selectin-, and L-selectin- | safety and efficacy | | [4] |
| (TBC1269) | dependent adhesion in humans in vitro and to exert anti- | of inhaled | | |
| | inflammatory effects in different animal models. | Bimosiamose | | |
| | 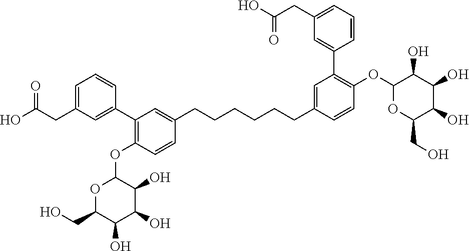 [SMILES: O=C(O)Cc1cccc(-c2cc(CCCCCCc3ccc(OC4O[C@H](CO)[C@H](O)[C@H](O)[C@@H]4O)c(-c4cccc(CC(=O)O)c4)c3)ccc2OC2O[C@H](CO)[C@H](O)[C@H](O)[C@@H]2O)c1] | for the treatment of patients with moderate to severe chronic obstructive pulmonary disease (COPD). Study to evaluate the effect of bimosiamose on ozone induced sputum Neutrophilia. Safety and efficacy study of Bimosiamose Cream to treat | II II | NCT00962481 NCT00823693 |
| | | psoriasis. | | |
| Rivipansel | Novel small molecule glycomimetic pan-selectin antagonist. [symbol] | Study of Intravenous | II, | NCT00911495 |
| (GMI- 1070) | 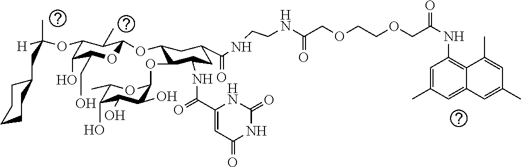 | GMI-1070 in adults with Sickle Cell Disease. Study of GMI-1070 for the treatment of Sickle Cell Pain Crisis. | III II | [5] NCT01119833 |

1. Ataga, K.I., et al., Crizanlizumab for the Prevention of Pain Crises in Sickle Cell Disease. N Engl J Med, 2017. 376(5): p. 429-439.
2. Bedard, P.W., et al., Characterization of the novel P-selectin inhibitor PSI-697 [2-(4-chlorobenzyl)-3-hydroxy-7,8,9,10-tetrahydrobenzo[h] quinoline-4-carboxylic acid] in vitro and in rodent models of vascular inflammation and thrombosis. J Pharmacol Exp Ther, 2008. 324(2): p. 497-506.
3. Park, I.Y., et al., Cylexin: a P-selectin inhibitor prolongs heart allograft survival in hypersensitized rat recipients. Transplant Proc, 1998. 30(7): p. 2927-8.
4. Mayr, F.B., et al., Effects of the pan-selectin antagonist bimosiamose (TBC1269) in experimental human endotoxemia. Shock, 2008. 29(4): p. 475-82.
5. Chang, J., et al., GMI-1070, a novel pan-selectin antagonist, reverses acute vascular occlusions in sickle cell mice. Blood, 2010. 116(10): p. 1779-86. [symbol]
indicates data missing or illegible when filed

The agent which is used to down-regulate the amount and/or activity of P-selectin may be formulated for crossing the blood brain barrier.

Exemplary methods for formulating the above described agents to enhance its penetration across the blood brain barrier are described in Yeini et al., Advanced Therapeutics, DOI: 10.1002/adtp.202000124.

Thus, for example, the agents can be formulated in nanoparticles such as liposome-based nanoparticles, amphiphilic micelles, dendrimers, inorganic nanoparticles and polymeric nanoparticles.

Specifically for the delivery of oligonucleotides, the use of cationic nanoemulsions modified biodegradable poly (B-Amino Ester) (PBAE), cell derived extracellular vesicles, spherical nucleic acid nanoparticles, may be considered to improve delivery to the brain.

Since the BBB restricts the passage of most therapeutic agents from the blood to the brain, receptor-mediated transcytosis can offer a non-invasive trafficking system to deliver targeted carriers into the brain parenchyma. In addition, this approach allows selective targeting of tumor cells within the brain tissue, thus reducing toxicity in other tissues and non-tumor cells in the brain. Examples of receptor-mediated approaches include manipulation of the apolipoprotein receptor, targeting of the epidermal growth factor receptor, transferrin receptor targeting, insulin receptor targeting and adhesion molecule targeting are all contemplated.

It will be appreciated that the P-selectin inhibitory agents may be directly attached to moieties that target the agent to the blood brain barrier or indirectly (e.g. P-selectin inhibitory agents may be comprised in a carrier which may be attached to the targeting moieties).

According to one aspect of the present invention, the P-selectin inhibitory agents are used (in conjunction with immunomodulatory agents) to treat brain-metastasized cancer.

Metastatic brain cancer (also called secondary brain tumors) is caused by cancer cells spreading (metastasizing) to the brain from a different part of the body.

According to a particular embodiment, the brain-metastasized cancer is selected from the group consisting of lung, breast, skin (melanoma), colon, kidney and thyroid gland.

As mentioned, the P-selectin inhibitor may be administered/co-formulated with an immunomodulatory agent. In one embodiment, the immunomodulatory agent is a nanoparticle which comprises a neoantigen peptide of glioblastoma (e.g. GL261). Methods of formulating such nanoparticles are disclosed in WO2020/136657, the contents of which are incorporated herein by reference.

In one embodiment, the immunomodulatory agent is a checkpoint inhibitor.

The phase “Checkpoint blockades” or “Checkpoint inhibitors” refers to a form of immunotherapy, meaning it aims to help the patient's own immune system fight cancer. It can use substances such as monoclonal antibodies or binding fragments thereof, which can be designed to target extremely specific molecules on cell surfaces. For example, the antibodies unblock a reaction that stops the immune system's natural attack on invading cancer cells. In another example, a ligand-receptor interaction that has been investigated as a target for cancer treatment is the interaction between the transmembrane programmed cell death 1 protein (PDCD1, PD-1; also known as CD279) and its ligand, PD-1 ligand 1 (PD-L1, CD274). In normal physiology PD-L1 on the surface of a cell binds to PD1 on the surface of an immune cell, which inhibits the activity of the immune cell. It appears that upregulation of PD-L1 on the cancer cell surface can allow them to evade the host immune system by inhibiting T cells that might otherwise attack the tumor cell. Antibodies that bind to either PD-1 or PD-L1 and therefore block the interaction can allow the T-cells to attack the tumor. In some alternatives, the checkpoint blockade therapeutics comprises anti-PD-1 antibodies or binding fragments thereof (e.g., monoclonal antibodies or humanized versions thereof or binding fragments thereof). In some alternatives, the checkpoint blockade therapeutics comprises PD-L1.

Examples of immunomodulatory agents include immunomodulatory cytokines, including but not limited to, IL-2, IL-15, IL-7, IL-21, GM-CSF as well as any other cytokines that are capable of further enhancing immune responses; immunomodulatory antibodies, including but not limited to, anti-CTLA4, anti-CD40, anti-41BB, anti-OX40, anti-PD1 and anti-PDL1; and immunomodulatory drugs including, but not limited to lenalidomide (Revlimid).

Exemplary anti-PD1 antibodies include Pembrolizumab (Keytruda), Nivolumab (Opdivo), Cemiplimab (Libtayo) and Dostarlimab (Jemperli).

According to a particular embodiment, the anti-PD1 antibody is Nivolumab.

Additional anti-PD1 antibodies include JTX-4014, Spartalizumab (PDR001), Camrelizumab (SHR1210), Sintilimab (IBI308), Tislelizumab (BGB-A317), Toripalimab (JS 001) INCMGA00012 (MGA012), AMP-224 AMP-514 (MEDI0680).

Other contemplated anti-cancer agents which may be administered to the subject in combination with the P-selectin inhibitor described herein include, but are not limited to Acivicin; Aclarubicin; Acodazole Hydrochloride; Acronine; Adriamycin; Adozelesin; Aldesleukin; Altretamine; Ambomycin; Amctantrone Acetate; Aminoglutethimide; Amsacrine; Anastrozole; Anthramycin; Asparaginase; Asperlin; Azacitidine; Azctepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifenc Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurca; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Mcturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

Examples of cancer include but are not limited to carcinoma, lymphoma, blastoma, sarcoma, and leukemia. Particular examples of cancerous diseases but are not limited to: Myeloid leukemia such as Chronic myelogenous leukemia. Acute myelogenous leukemia with maturation. Acute promyelocytic leukemia, Acute nonlymphocytic leukemia with increased basophils, Acute monocytic leukemia. Acute myelomonocytic leukemia with cosinophilia; Malignant lymphoma, such as Birkitt's Non-Hodgkin's; Lymphoctyic leukemia, such as Acute lumphoblastic leukemia. Chronic lymphocytic leukemia; Myeloproliferative diseases, such as Solid tumors Benign Meningioma, Mixed tumors of salivary gland, Colonic adenomas; Adenocarcinomas, such as Small cell lung cancer, Kidney, Uterus, Prostate, Bladder, Ovary, Colon, Sarcomas, Liposarcoma, myxoid, Synovial sarcoma, Rhabdomyosarcoma (alveolar), Extraskeletel myxoid chonodrosarcoma, Ewing's tumor; other include Testicular and ovarian dysgerminoma, Retinoblastoma, Wilms' tumor, Neuroblastoma, Malignant melanoma, Mesothelioma, breast, skin, prostate, and ovarian.

According to a particular embodiment, the cancer is a solid tumor.

Exemplary cancers that can be treated using the combined therapy (i.e. immunomodulatory agent+P-selectin inhibitors) include pancreatic cancer, lung cancer, breast cancer, primary melanoma and kidney cancer.

The P-selectin inhibitors may be co-formulated with the immunomodulatory agents described herein, or may be provided as separate compositions to the subject.

Thus, each agent included in the combination can be formulated separately for use in combination. The drugs are said to be used “in combination” when, in a recipient of both drugs, the effect of one drug enhances or at least influences the effect of the other drug.

The two agents in the combination cooperate to provide an effect on target cells that is greater than the effect of either drug alone. This benefit manifests as a statistically significant improvement in a given parameter of target cell effect. In embodiments, the improvement resulting from treatment with the drug combination can manifest as an effect that is at least additive and desirably synergistic, relative to results obtained when only a single agent is used.

In use, each drug in the combination can be formulated as it would be for monotherapy, in terms of dosage size and form and regimen. In this regard, the synergy resulting from their combined use may permit the use of somewhat reduced dosage sizes or frequencies, as would be revealed in an appropriately controlled clinical trial.

According to one embodiment, the P-selectin inhibitor and the immunomodulatory agent are administered concomitantly.

According to another embodiment, the P-selectin inhibitor and the immunomodulatory agent are administered sequentially, wherein the first agent is used, for example, 30 minutes, 1 hour, 6 hours, 12 hours, 24 hours, 48 hours, 72 hours, 96 hours, a week, a month or more after the second agent. Such a determination is well within the capacity of one of skill in the art. In another embodiment, the P-selectin inhibitor and the immunomodulatory agent are administered sequentially, wherein the second agent is used, for example, 30 minutes, 1 hour, 6 hours, 12 hours, 24 hours, 48 hours, 72 hours, 96 hours, a week, a month or more after the first agent.

The P-selectin inhibitors (and immunomodulatory agents) of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the P-selectin inhibitor accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into the brain of a patient.

Pharmaceutical compositions of some embodiments of the invention may be manufactured by processes well known in the art, e.g., by means of conventional mixing, dissolving, granulating, dragcc-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multi-dose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (P-selectin inhibitor) effective to prevent, alleviate or ameliorate symptoms of a disorder (e.g., P-selectin inhibitor) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide levels (e.g. brain levels) of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

For any preparation used in the methods of the invention, the dosage or the therapeutically effective amount can be estimated initially from in vitro and cell culture assays. For example, a dose can be formulated in animal models to achieve a desired concentration or titer. Such information can be used to more accurately determine useful doses in humans. Since administration of the disclosed combination is expected to produce improved results over the administration of single agents, the therapeutically effective dose of each of the agents in the combined treatment may be for example less than 50%, 40%, 30%, 20% or even less than 10% the of the FDA approved dose.

For example, therapeutically effective dose of the immunomodulatory agent (e.g. immunomodulatory antibody) in the combined treatment may be for example less than 50%, 40%, 30%, 20% or even less than 10% the of the FDA approved dose. Conversely, the therapeutically effective dose of the P-selectin inhibitor in the combined treatment may be for example less than 50%, 40%, 30%, 20% or even less than 10% the of the FDA approved dose.

Depending on the severity and responsiveness of the condition to be treated, dosing can be of a single or a plurality of administrations, with course of treatment lasting from several days to several weeks or until cure is effected or diminution of the disease state is achieved.

According to another aspect of the present invention there is provided a method of treating glioblastoma or a brain metastasized melanoma in a subject in need thereof comprising administering to the subject a therapeutically effective amount of Crizanlizumab and an anti-PD1 antibody, thereby treating the glioblastoma or brain metastasized melanoma.

As used herein, the term “glioblastoma” (GBM), also called glioblastoma multiforme or “grade IV astrocytoma” according to WHO classification refers to a central nervous system primary tumor derived from glial cells. GBM is one of the deadliest human cancers with an incidence of about 3.5/100,000 per year worldwide (Cloughesy, T. F., W. K. Cavence, and P. S. Mischel, Glioblastoma: from molecular pathology to targeted treatment. Annu Rev Pathol, 2014. 9: p. 1-25). Despite the aggressive standard of care currently used including surgery, chemo- and radiotherapy, the prognosis remains very poor with about 15 months overall survival.

According to a particular embodiment, the glioblastoma is at an early stage (e.g. when the tumor is of a diameter of less than 14 mm).

An exemplary treatment regimen for the treatment of cancer (e.g. glioblastoma) is as follows:
1. Treat subject with Crizanlizumab (e.g. 1-20 mg/kg, more specifically about 5 mg/kg)+Anti-PD1 antibody (e.g. nivolumab) (e.g. 1-20 mg/kg, more specifically about 3 mg/kg) once every two weeks;
2. After two or three rounds of (1), treat subject with Crizanlizumab (e.g. 1-20 mg/kg, more specifically about 5 mg/kg) once every four weeks. At stage 2, the anti-PD1 antibody (e.g. nivolumab) (1-20 mg/kg, more specifically about 3 mg/kg) may be administered once every two weeks or once every four weeks.

According to a particular embodiment, the active agent is administered following resection of the glioblastoma tumor.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

### Method of Treating

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

### Treatment Regimen

As used herein the phrase “treatment regimen” refers to a treatment plan that specifies the type of treatment, dosage, schedule and/or duration of a treatment provided to a subject in need thereof (e.g., a subject diagnosed with a pathology). The selected treatment regimen can be an aggressive one which is expected to result in the best clinical outcome (e.g., complete cure of the pathology) or a more moderate one which may relief symptoms of the pathology yet results in incomplete cure of the pathology. It will be appreciated that in certain cases the more aggressive treatment regimen may be associated with some discomfort to the subject or adverse side effects (e.g., a damage to healthy cells or tissue). The type of treatment can include a surgical intervention (e.g., removal of lesion, diseased cells, tissue, or organ), a cell replacement therapy, an administration of a therapeutic drug (e.g., receptor agonists, antagonists, hormones, chemotherapy agents) in a local or a systemic mode, an exposure to radiation therapy using an external source (e.g., external beam) and/or an internal source (e.g., brachytherapy) and/or any combination thereof. The dosage, schedule and duration of treatment can vary, depending on the severity of pathology and the selected type of treatment, and those of skills in the art are capable of adjusting the type of treatment with the dosage, schedule and duration of treatment.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials

DMEM, fetal bovine serum (FBS), L-glutamine, penicillin, streptomycin, mycoplasma detection kit, EZ-RNA II total RNA isolation kit and fibronectin (1 mg/ml, dilution: 1:100) were purchased from Biological Industries Ltd. (Kibbutz Beit HaEmek, Israel). Percoll medium (Cat. No. p4937) and all other chemical reagents, including salts and solvents, were purchased from Sigma-Aldrich (Rehovot, Israel). Milli-Q water was prepared using a Millipore water purification system. Amicon Ultra Centrifugal Filters; molecular weight cut-off (MWCO) 5 or 3 kDa and Poly-L-Lysine (PLL) (Cat. No. A-005-C; 0.1 mg/ml) were purchased from Merck Millipore (Burlington, Massachusetts, USA). The qScript™ cDNA Synthesis Kit was purchased from Quantabio (Beverly, MA, USA). Fast SYBR™ green Master Mix was purchased from Applied Biosystems (California, USA). Collagenase IV, Dispase II (neutral protease) and DNase I were purchased from Worthington Biochemical Corporation (NJ, USA). RBC lysis solution (Cat. No. 420301) was purchased from BioLegend (San Diego, California, USA). MACS MS magnetic columns for cell separation (Cat. No. 130-042-201), CD11b MicroBeads for cell isolation (Cat. No. 130-093-634) and CD45 (TIL) MicroBeads for cell isolation (Cat. No. 130-110-618) were purchased from Miltenyi Biotec (Bergisch Gladbach, Germany). SELP inhibitor (SELPi) KF38789 (Cat. No. 2748) was purchased from Tocris BioScience (Bristol, United Kingdom). Recombinant human SELP (Cat. No. ADP3; Lot. No. ARL6019071), Recombinant murine SELP (rSELP) (Cat. No. 10094-PS; Lot. No. DKLJ0118111), Human SELP ELISA kit (Cat. No. DPSE00), Total NO/Nitrite/Nitrate Immunoassay (Cat. No. KGE001), Mouse XL Cytokine Array Kit (Cat. No. ARY028), Human Cytokine Array kit (Cat. No. ARY005B), anti-human PSGL-1 neutralizing antibody (Cat. No. MAB3345; Lot. No. CLYK0120111; Clone 688102), and anti-human SELP neutralizing antibody (Cat. No. AF137; Lot. No. FBX0518051) were purchased from R&D Systems (Minneapolis, Minnesota, USA). Human L-507 cytokine array kit (Cat. No. AAH-BLM-1A-4; Lot No. 102920 009) was purchased from RayBiotech (Norcross, Georgia, United States). Anti-human/mouse CD44 neutralizing antibody (Cat. No. NBP2-2530; Lot No. VC289186) was purchased from Novus (Colorado, USA). Anti-murine PSGL-1 neutralizing antibody (Cat. No. BE0188; Lot No. 676818M2) was purchased from Bio X Cell (Massachusetts, USA). MEBCYTO Apoptosis Kit, was purchased from MBL International (UK), Recombinant murine GM-CSF (Cat. No. 315-03-50 ug; Lot. No. 091855) was purchased from PeproTech (Rehovot, Israel). Latex beads for phagocytosis assays (Cat. No. L4655) were purchased from Sigma-Aldrich (Rehovot, Israel). ProLong® Gold mounting with DAPI (Cat. No. p36935) and Hoechst 33342 (Cat. No. H3570) were purchased from Invitrogen (Carlsbad, California, USA). Mayer's Hematoxylin solution (Cat. No. 05-06002) and Eosin Y solution (Cat. No. 05-10002) were purchased from Bio-Optica (Milano, Italy). Anti-PD-1 antibody was purchased from ichorbio Ltd (Wantage, USA, Cat. ICH1132; Lot. 1220L520). Immunostaining antibodies: Mouse anti-human SELP (Cat. No. BBA1; Lot. No. APB081704; Clone BBIG-E; Dilution 1:30) was purchased from R&D Systems (Minneapolis, Minnesota, USA). Mouse anti-mouse SELP (Cat. No. 148302; Lot No. B186735; Clone RMP-1; Dilution 1:50) was purchased from BioLegend (San Diego, California, USA). Goat anti-mouse Alexa Fluor® 647 (Cat. No. ab15115; Lot. No. GR309891-3; Dilution 1:300). Flow cytometry antibodies: Mouse anti-human SELP (Cat. No. BBA1; Lot. No. APB081704; Clone BBIG-E; Dilution 1:20), Mouse IgGI isotype control (Cat. No. mab002; Dilution 1:20) were purchased from R&D Systems (Minneapolis, Minnesota, USA).

### Methods

### Cell Culture

Metastatic melanoma 131/4-5B1 cells were cultured in RPMI medium supplemented with 10% FBS, 100 IU/mL Penicillin, 100 μg/mL Streptomycin, 12.5 IU/mL Nystatin, 2 mM L-glutamine. Primary melanoma A375 cells (ATCC, USA) were cultured in RPMI medium supplemented with 10% FBS, 100 IU/mL Penicillin, 100 μg/mL Streptomycin, 12.5 IU/mL Nystatin, 2 mM L-glutamine, 1 mM sodium pyruvate, and 25 mM HEPES. Primary melanoma WM115 cells (ECACC, Porton Down, Salisbury, UK) were cultured in MEM medium supplemented with 10% FBS, 100 IU/mL Penicillin, 100 μg/mL Streptomycin, 12.5 IU/mL Nystatin, and 2 mM L-glutamine, ImM sodium pyruvate and 1x of MEM NAA. Primary melanoma B16-F10 cells (ATCC, USA) were cultured in DMEM supplemented with 10% FBS, 100 U/mL Penicillin, 100 μg/mL Streptomycin, 12.5 IU/mL Nystatin, and 2 mM L-glutamine. Primary melanoma B2905 were grown in RPMI supplemented with 10% FBS, 100 U/mL Penicillin, 100 μg/mL Streptomycin, 12.5 IU/mL Nystatin, 25 mM HEPES, and 2 mM L-glutamine. Primary melanoma Mel-ret cells (kindly provided by Neta Erez) were grown in RPMI supplemented with 10% FBS, 100 U/mL Penicillin, 100 μg/mL Streptomycin, 12.5 IU/mL Nystatin, 1 mM sodium pyruvate, and 2 mM L-glutamine. Primary melanoma D4M.3A cells (kindly provided by David W. Mullis) were grown in Advanced DMEM supplemented with 5% FBS, 100 IU/mL Penicillin, 100 μg/mL Streptomycin, 12.5 IU/mL Nystatin, and 2 mM Glutamax. All melanoma cell lines were labeled with pQC-mCherry retroviral particles, as previously described.

### Primary Murine Microglia Cell Isolation

Brains resected from healthy, 5-8 week old C57BL/6 mice, were chopped and incubated with 1 mg/ml Collagenase IV, 2 mg/ml Dispase II (neutral protease), and 0.02 U/ml DNase I for 50 min at 37° C. Red blood cells (RBC) were lysed with RBC lysis solution followed by a Percoll gradient for myelin separation. The resultant cell-suspension was then incubated with CD11b microbeads and the desired population was isolated on MACS MS magnetic columns. Murine microglia were then seeded on poly-L-Lysine (PLL)-coated plates in microglia medium (ScienCell, California, USA).

### Splenocytes Isolation

Splenocytes were freshly isolated from the spleens of healthy 7-11 weeks old C57BL/6 mice. Spleens were mashed and passed through a 70 μm nylon strainer, followed by RCB lysis. Flasks were coated with anti-CD38 (Cat. 100340; Lot. B302116; Clone 145-2C11) antibody, and splenocytes (30×106 cells/75 cm2 flask) were incubated with 2 μg/ml anti-CD28 (Cat. 102116; Lot. B331922; Clone 3751), and 10 U/ml rhIL-2 for 6 days.

### Flow Cytometry

For flow cytometry assays, cells were harvested using a cell scraper, and were then washed with PBS followed by additional washes with PBS supplemented with 1% BSA and 5 mM EDTA (FACS buffer). Tumor spheroids were recovered from Matrigel using Cell Recovery Solution (Corning) and washed with FACS buffer. To assess P-Selectin expression, cells were then incubated with anti-human, or anti-mouse P-Selectin antibody for 1 h on ice, and then were washed, and incubated with Alexa-488 labeled anti-mouse IgG binding protein for 1 h on ice.

### Co-Culture Proliferation Assay

Primary murine microglia were seeded in a 24 well plate (Corning) at different concentrations, and the same amount of mCherry-labeled murine Mel-ret (1:4, 1.5:1, 3:1, 6:1 ratios), were added 24-72 h post seeding. The plates were then incubated for 96 h (37° C.; 5% CO2) in microglia medium and proliferation of fluorescently-labeled cells was measured by the IncuCyte Zoom Live cell analysis system (Essen Bioscience). Melanoma cells in microglia medium were used as a control.

### Splenocytes and Melanoma Spheroids Co-Culture

Tumor spheroids were prepared from D4M m-cherry-labeled. D4M spheroids were prepared by seeding 500 cells per well of U-shape bottom, low attachment 96 well plate. Splenocytes were harvested from C57BL/6J mice and seeded in 96 well plate containing D4M spheroids in ratio of 1:50, and incubated with complete RPMI supplemented with 10% (v/v) FBS, 1% (v/v) PEST, 1% (v/v) HEPES, 1% (v/v) sodium pyruvate and 0.1% (v/v) 2-Mercaptoethanol. Co-culture cells were treated with SELP inhibitors KF38789 (Tocris, 0.5 μM), or PSI-697 (10 μM) with or without anti-PD-1 antibody (0.5 mg/ml). The growth of fluorescently-labeled melanoma spheroids was measured by the IncuCyte Zoom Live cell analysis system (Essen Bioscience).

### RNA Isolation

EZ-RNA II total RNA isolation kit (Biological Industries Ltd., Israel) was used to isolate total RNA, according to the manufacturer's protocol. Briefly, samples were lysed with 0.5 ml Denaturing Solution/10 cm2 culture plate. Water saturated phenol was then added, and the samples were centrifuged. Isopropanol was added to precipitate the RNA and the centrifuged RNA pellet was washed with 75% ethanol, centrifuged, and re-suspended with ultra-pure double distilled water. RNA concentration was evaluated using a NanoDrop® ND-1000 Spectrophotometer according to the manufacturer's V3.5 User's Manual (Nano-Drop Technologies, Wilmington, DE).

cDNA Synthesis

qScript™ cDNA synthesis kit for RT-PCR was used to synthesize cDNA, according to the manufacturer's protocol. Briefly, 1 μg of total RNA sample was mixed with qScript Reverse Transcriptase, dNTPs, and nuclease free water. The reaction tube was then incubated at 42° C. for 30 min and heated at 85° C. for 5 min to stop cDNA synthesis.

### Real-Time PCR

The expression level of target genes was assessed by SYBR green real-time PCR (StepOne plus, Life Technologies) and normalized to GAPDH housekeeping gene.

### Animals and Ethics Statement

Animals were housed in the Tel Aviv University animal facility. All experiments received ethical approval by the animal care and use committee (IACUC) of Tel Aviv University (protocol no. 01-16-054 and no. 01-21-006) and conducted in accordance with NIH guidelines.

### Animal Models

To generate primary melanoma tumors, murine or human cells-(0.5×106 cells/100 μl) were inoculated intradermal (i.d.) in immunocompetent C57BL/6 mice or 6-8 weeks old male immunocompromised SCID mice, respectively. To generate intracranial melanoma tumors, murine or human cells (1.5×104 cells/2 μl) were intracranially inoculated stereotactically to the striatum of 8 to 10-week-old immunocompetent C57BL/6 mice or 6-8 weeks old male immunocompromised SCID mice, respectively. Mice body weight change was monitored twice a week, and tumor growth was measured using 4.7 T MRI (MR Solutions, UK). Mice were euthanized and brains were then harvested for further immunostaining and flow cytometry analysis.

### Frozen OCT Tissue Fixation

Tumor bearing mice were anesthetized with an IP injection of ketamine (150 mg/kg) and xylazine (12 mg/kg) and perfused with PBS followed by 4% Paraformaldehyde (PFA). Brains were harvested, and incubated with 4% PFA for 4 h, followed by 0.5 M of sucrose (BioLab) for 1 h, and 1 M sucrose overnight (ON). The brains were then embedded in optimal cutting temperature (OCT) compound (Scigen) on dry ice and stored at −80° C.

### Immunostaining

OCT embedded tumor samples were cut into 5 μm thick sections. Staining was performed using BOND RX autostainer (Leica). Sections were stained by hematoxylin and cosin (H&E) and immunostained for P-selectin. Prior to antibody incubation, slides were incubated with 10% goat serum in PBS x1+0.02% Tween-20, for 30 min to block non-specific binding sites. Slides were incubated with primary antibodies for 1 h, and then washed and incubated with secondary antibodies for an additional 1 h. They were then washed and treated with ProLong® Gold mounting with DAPI before being covered with coverslips. Stained samples were imaged using the EVOS FL Auto cell imaging system (ThermoFisher Scientific). At least three fields of each individual sample were imaged and quantified using ImageJ 1.52v software. Quantification of positive staining was performed by measuring the total area stained in each image following background subtraction, using single color images representing the correlated marker.

### Statistical Analysis

Data are expressed as mean ±standard deviation (s.d.) for in vitro assays or ±standard error of the mean (s.c.m.) for in vivo assays. Statistical significance was determined using an unpaired, two-sided t-test when comparing between two groups, and multiple comparisons ANOVA test when comparing more than two groups. P<0.05 was considered statistically significant. For Kaplan-Meier survival curves, p values were determined using log rank test. For in vivo tumor growth curves, p values were determined using one-way ANOVA, Dunn's test, or Holm-Sidak's test. Statistical analysis was performed using GraphPad Prism 8.

### Results

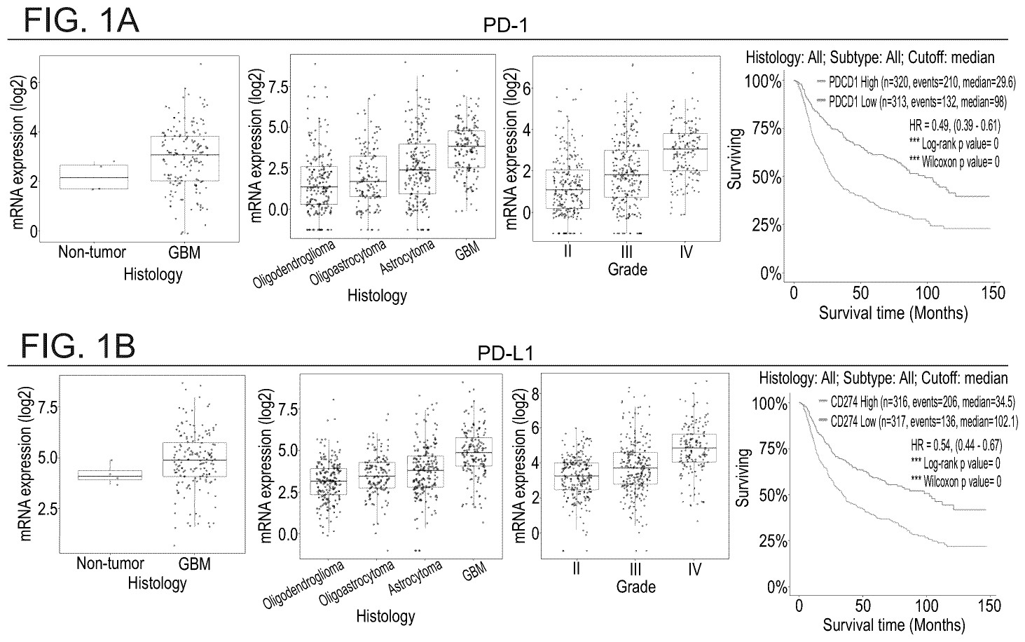

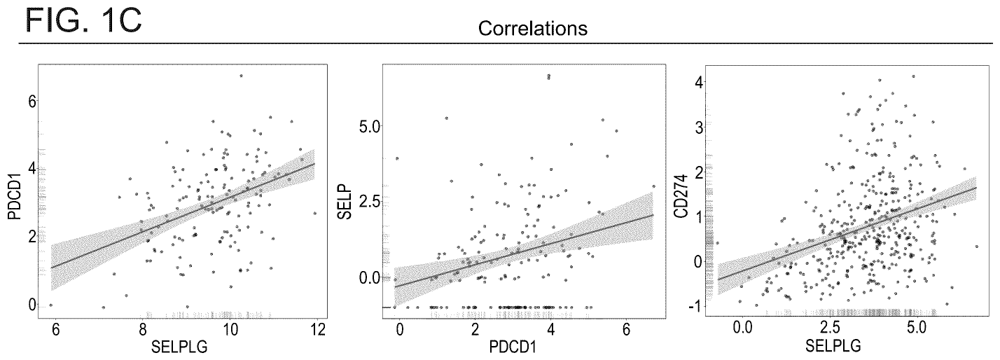

GB tumors are characterized by a highly suppressive tumor microenvironment. To investigate whether PD-1 and its ligand PD-L1 are highly expressed in GB tumors, GB patients data bases were explored using the GlioVis data portal (Bowman, R. L et al Neuro-oncology 2017, 19 (1), 139-141). The analysis showed high expression of both PD-1 (PDCD1) and PD-L1 (CD274) in GB samples compared to healthy brain and low-grade gliomas samples. Moreover, it was found that their expression is negatively correlated with patient survival (FIGS. 1A-B), indicating the role of PD-1/PDL-1 axis in GB progression. It was further found that PSGL-1 (SELPLG) expression is positively correlated with both PD-1 and PDL-1 expression, and that SELP expression is positively correlated with PD-L1 expression in GB patient tissues. This may suggest that immunosuppressive microglia/macrophages cells express both PSGL-1 and PD-L1 and facilitate PD-1 expression by T cells, and that GB cells express SELP and PD-L1. These results may explain the lack of therapeutic benefit of anti-PD-1 in GB patients as a monotherapy.

In order to assess whether SELP and PD-1 axes are relevant in other brain tumors, collected FFPE samples of normal human brain were collected together with patient samples of melanoma, breast, lung and CRC brain metastasis (BM) as well as patient diffuse intrinsic pontine glioma (DIPG) samples. Immunostaining revealed high expression of SELP and PD-L1 in all samples, high expression of PSGL-1 in DIPG and BM of melanoma, breast and lung, and high expression of PD-1 in melanoma and lung BM (FIGS. 2A-B). Moreover, we found high expression of SELP in primary melanoma, breast, lung, and pancreatic ductal adenocarcinoma (PDAC) patient samples. PSGL-1 was found to be highly expressed in primary melanoma, breast and lung samples, PD-1 and PD-L1 in primary melanoma, PDAC and lung samples as well (FIGS. 3A-B).

In order to investigate the role of microglia in cancer brain metastasis, several melanoma brain metastasis (MBM) mouse models were established. Using Iba-1 immunostaining, activated microglia were identified in the tumor site in all the different models and in MBM patient formalin-fixed paraffin-embedded (FFPE) samples (FIG. 4A). To evaluate the effect of microglia on MBM progression, primary murine microglia were co-cultured with the murine RET MBM cell line and cancer cell proliferation was observed. A direct correlation between the increased proliferation rate of murine RET MBM cells and the concentration of murine microglia in the culture was noted (FIG. 4B).

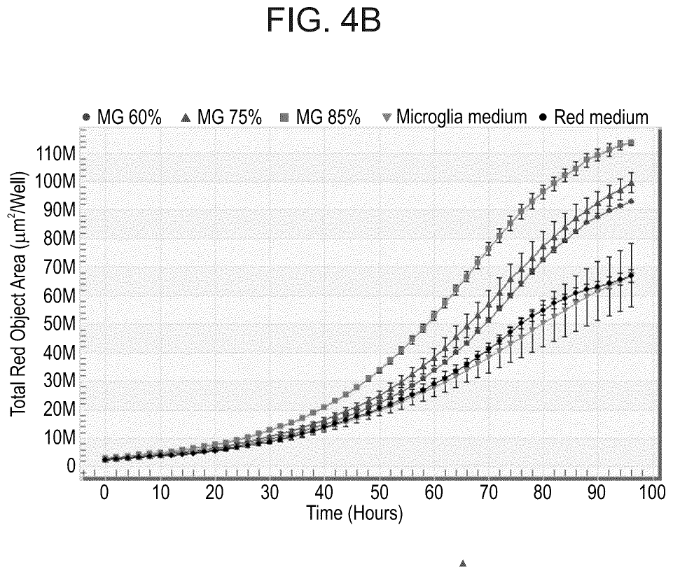

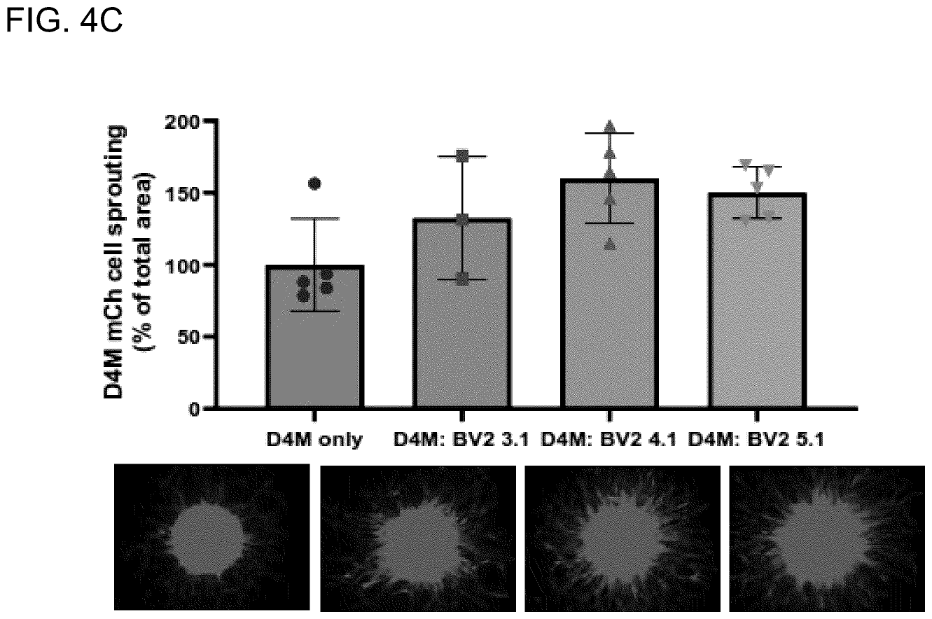

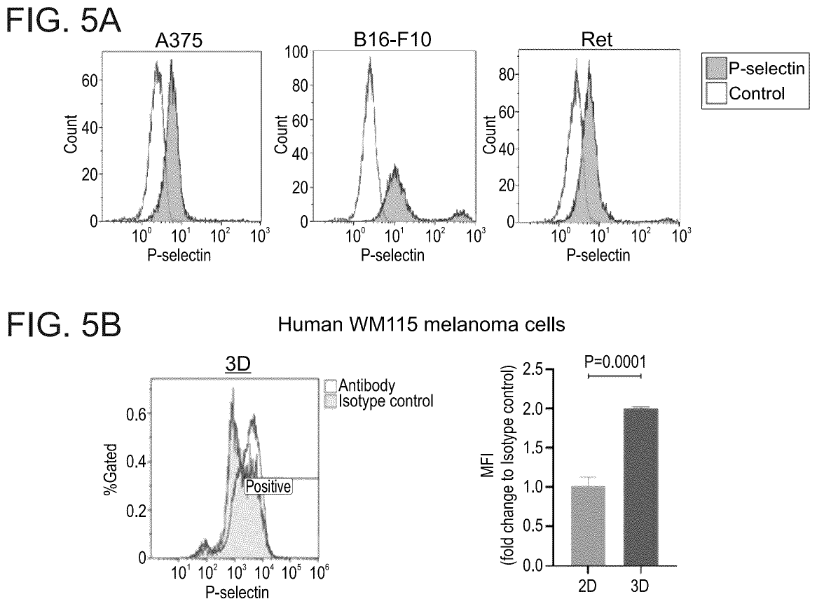

In addition, 3D in vitro models of melanoma were established using the human WM115 and the murine D4M.3A melanoma cell lines. Increased invasion of D4M.3A spheroids was observed when in the presence of microglia in the spheroids (FIG. 4C), as well as higher expression of SELP compared to melanoma cells grown on 2D plastic culture dishes (FIGS. 5A-C). This expression was increased over time following Matrigel seeding of the D4M.3A spheroids (FIG. 5C).

In order to evaluate the therapeutic potential of a combined treatment of SELP inhibitors (SELPi) and anti-PD-1 antibody, D4M.3A tumor spheroids were co-cultured with primary murine microglia. Following spheroids formation, freshly isolated mouse splenocytes were added together with the different treatments and cancer cell killing by the splenocytes was monitored. The results showed synergistic effects of SELPi treatment with anti-PD-1 antibody in the spheroids composed of both melanoma cells and microglia, while only minor effects were observed in D4M.3A spheroids without microglia (FIGS. 6A-B). This indicates that microglia are required in order to induce cancer cell killing by the splenocytes following SELP and PD-1 inhibition.

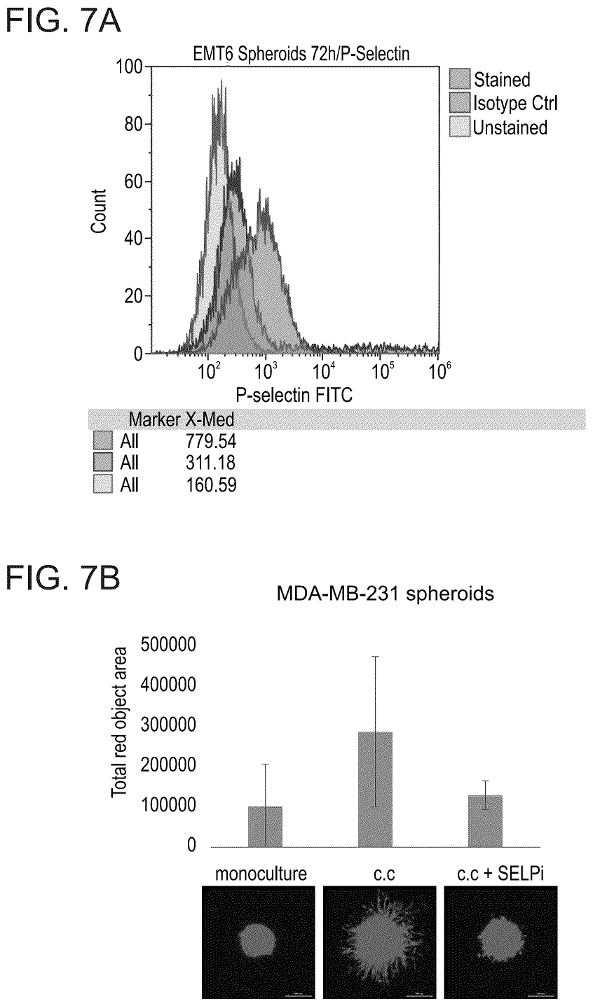

Validating these results in additional cancer models, we found high expression of SELP in the EMT-6 murine breast cancer cell line (FIG. 7A). Moreover, using human astrocyte-MDA-MB-231 breast cancer multi-cellular 3D spheroids, we manage to reduce cancer cell invasion following SELP inhibition (FIG. 7B). This may suggest that SELP is important for the interactions of breast cancer cells with astrocytes as well.

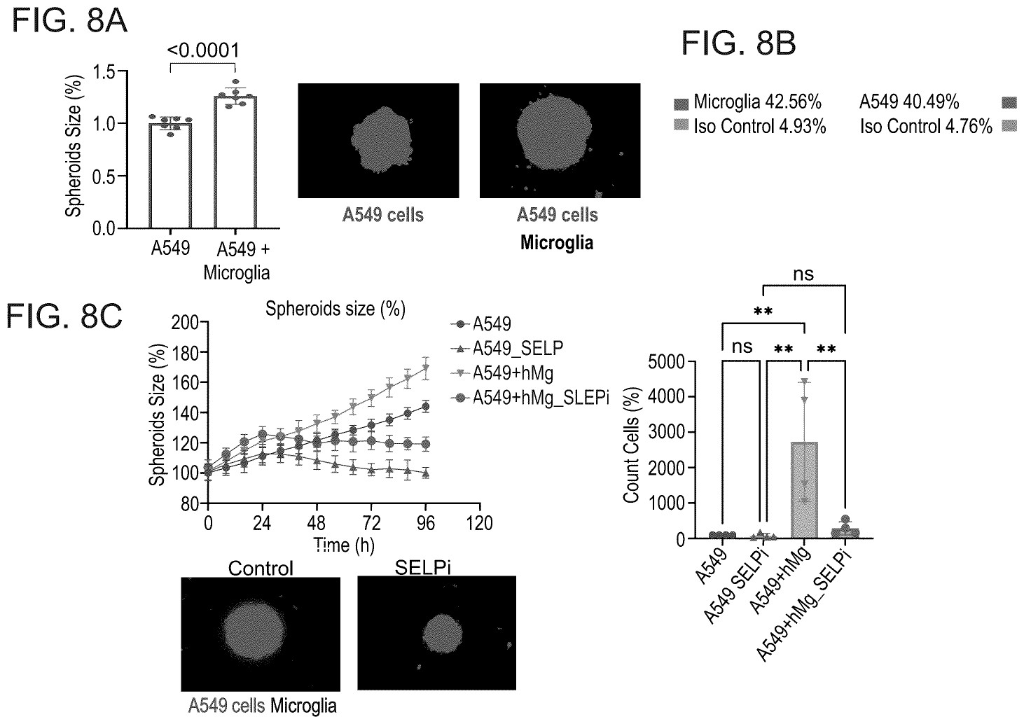

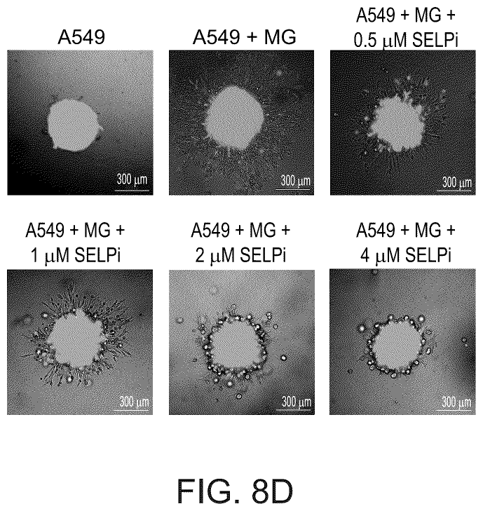

When looking at lung cancer cells, an increased invasion of human A549 spheroids was observed when co-cultured with human microglia (FIG. 8A). Flow cytometry analysis of these spheroids showed high expression of SELP and PSGL-1 in A549 cells and human microglia, respectively (FIG. 8B). Indeed, SELP inhibition reduced spheroid growth and invasion in A549-microglia 3D models (FIGS. 8C-D).

Taken together, the results showed the potential therapeutic benefit of combining SELP inhibition with other immunomodulators, such as anti-PD-1, for both primary and secondary brain malignancies.

Although the invention has been described in conjunction with specific embodiments thereof, it is evident that many alternatives, modifications, and variations will be apparent to those skilled in the art. Accordingly, it is intended to embrace all such alternatives, modifications, and variations that fall within the spirit and broad scope of the appended claims.

It is the intent of the applicant(s) that all publications, patents, and patent applications referred to in this specification are to be incorporated in their entirety by reference into the specification, as if each individual publication, patent, or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of treating a cancer selected from the group consisting of brain-metastasized cancer, pancreatic cancer, lung cancer, breast cancer, primary melanoma and kidney cancer in a subject in need thereof comprising administering to the subject a therapeutically effective amount of an agent that specifically decreases an amount and/or activity of P-selectin and an immunomodulatory agent, thereby treating the cancer.

2. The method of claim 1, wherein said brain-metastasized cancer is brain-metastasized melanoma, brain-metastasized breast cancer, brain-metastasized lung cancer, or brain-metastasized colorectal cancer.

3. The method of claim 1, wherein said agent that specifically decreases an amount and/or activity of P-selectin specifically binds to P-selectin or a polynucleotide encoding said P-selectin.

4. The method of claim 1, wherein said agent that specifically decreases an amount and/or activity of P-selectin binds to P-Selectin glycoprotein ligand-1 (PSGL-1) or a polynucleotide encoding said PSGL-1.

5. The method of claim 1, wherein said immunomodulatory agent comprises an immunomodulatory antibody.

6. The method of claim 5, wherein said immunomodulatory antibody is selected from the group consisting of anti-CTLA4, anti-CD40, anti-41BB, anti-OX40, anti-PD1, anti-PDL1, anti-LAG3, anti-IDO and anti-TIGIT.

7. The method of claim 1, wherein said agent that specifically decreases an amount and/or activity of P-selectin is an inhibitory antibody that binds to and inhibits said P-selectin.

8. The method of claim 7, wherein said inhibitory antibody is Crizanlizumab or Inclacumab.

9. The method of claim 8, wherein a dose of said Crizanlizumab is about 5 mg/kg once every two weeks or once every four weeks.

10. The method of claim 1, wherein said agent that specifically decreases an amount and/or activity of P-selectin is a small molecule agent.

11. The method of claim 1, wherein said agent that specifically decreases an amount and/or activity of P-selectin is a polynucleotide agent.

12. The method of claim 1, wherein said agent that specifically decreases an amount and/or activity of P-selectin is co-formulated with said immunomodulatory agent.

13. The method of claim 1, wherein said agent that specifically decreases an amount and/or activity of P-selectin is comprised in a nanoparticle.

14. The method of claim 13, wherein said nanoparticle is attached to a targeting moiety that increases delivery across the blood brain barrier.

15. The method of claim 1, wherein said agent that specifically decreases an amount and/or activity of P-selectin is attached to a targeting moiety that increases delivery across the blood brain barrier.

16. A method of treating glioblastoma in a subject in need thereof comprising administering to the subject a therapeutically effective amount of Crizanlizumab and an anti-PD1 antibody, thereby treating the glioblastoma or brain metastasized melanoma.

17. The method of claim 16, wherein said Crizanlizumab is delivered once every two weeks or once every four weeks at a dose of about 5 mg/kg.

18. The method of claim 16, wherein said anti-PD1 antibody is selected from the group consisting of Pembrolizumab (Keytruda), Nivolumab (Opdivo), Cemiplimab (Libtayo) and Dostarlimab (Jemperli).

19. The method of claim 16, wherein said anti-PD1 antibody is Nivolumab.

20. The method of claim 16, wherein said Crizanlizumab and said anti-PD1 antibody are initially administered once every two weeks for a duration of at least four weeks and optionally wherein a dose of said anti-PD1 antibody, is about 3 mg/kg once every two weeks.
